(19) United States
(12) Patent Application Publication
(10) Pub. No.: US 2022/0361733 A1
(43) Pub. Date: Nov. 17, 2022

(54) ENDOSCOPIC EXAMINATION SUPPORTING APPARATUS, ENDOSCOPIC EXAMINATION SUPPORTING METHOD, AND NON-TRANSITORY RECORDING MEDIUM RECORDING PROGRAM

(71) Applicant: OLYMPUS CORPORATION, Tokyo (JP)

(72) Inventors: Naoki FUKATSU, Tokyo (JP); Ryo TOJO, Tokyo (JP); Hiromasa FUJITA, Tokyo (JP)

(73) Assignee: OLYMPUS CORPORATION, Tokyo (JP)

(21) Appl. No.: 17/867,759

(22) Filed: Jul. 19, 2022

Related U.S. Application Data
(63) Continuation of application No. PCT/JP2020/001738, filed on Jan. 20, 2020.

(51) Int. Cl. A61B 1/00; G06T 7/00; A61B 5/06

(52) U.S. Cl. CPC A61B 1/00055; G06T 7/0012; A61B 5/065; A61B 1/0002; A61B 1/00009; A61B 1/00045; G06T 2207/10068; A61B 1/04

(57) ABSTRACT

An endoscopic examination supporting apparatus includes at least one processor including hardware. The processor acquires insertion shape information indicating an insertion shape of an insertion section of an endoscope inserted into a subject, evaluates, based on the insertion shape information, a procedure including inserting operation for the insertion section performed by a user who operates the endoscope during an endoscopic examination, and generates procedure evaluation information.

### CROSS REFERENCE TO RELATED APPLICATION

This application is a continuation application of PCT/JP2020/001738 filed on Jan. 20, 2020, the entire contents of which are incorporated herein by this reference.

### BACKGROUND OF THE INVENTION

### 1. Field of the Invention

The present invention relates to an endoscopic examination supporting apparatus, an endoscopic examination supporting method, and a non-transitory recording medium recording a program.

### 2. Description of the Related Art

In an endoscope field, there has been proposed a technique for supporting various kinds of operation performed by a user such as inserting operation for inserting a flexible elongated insertion section into a depth in a subject.

More specifically, for example, International Publication No. 2016/135966 discloses an apparatus that detects a shape of an insertion section of an endoscope and generates, based on the shape of the insertion section, operation support information indicating a state of the insertion section.

### SUMMARY OF THE INVENTION

An endoscopic examination supporting apparatus according to an aspect of the present invention includes at least one processor including hardware. The processor acquires insertion shape information indicating an insertion shape of an insertion section of an endoscope inserted into a subject, evaluates, based on the insertion shape information, a procedure including inserting operation for the insertion section performed by a user who operates the endoscope during an endoscopic examination, and generates procedure evaluation information.

An endoscopic examination supporting method according to an aspect of the present invention includes: acquiring insertion shape information indicating an insertion shape of an insertion section of an endoscope inserted into a subject; and evaluating, based on the insertion shape information, a procedure including inserting operation for the insertion section performed by a user who operates the endoscope during an endoscopic examination and generating procedure evaluation information.

A non-transitory recording medium recording a program according to an aspect of the present invention records a program for causing a computer to execute processing for: acquiring insertion shape information indicating an insertion shape of an insertion section of an endoscope inserted into a subject; and evaluating, based on the insertion shape information, a procedure including inserting operation for the insertion section performed by a user who operates the endoscope during an endoscopic examination and generating procedure evaluation information.

### BRIEF DESCRIPTION OF THE DRAWINGS

FIG. 1 is a diagram showing a configuration of a main part of an endoscope system including an endoscopic examination supporting apparatus according to an embodiment;

FIG. 2 is a block diagram for explaining an example of a configuration of an endoscope system according to a first embodiment;

FIG. 3 is a block diagram for explaining an example of a configuration of an examination-situation-information acquiring unit according to the first embodiment;

FIG. 4 is a diagram for explaining an example of table data used for processing performed in the endoscope system according to the first embodiment;

FIG. 5 is a block diagram for explaining an example of a configuration of a procedure evaluating unit according to the first embodiment;

FIG. 6 is a flowchart for explaining an example of processing performed in the endoscope system according to the first embodiment;

FIG. 7 is a diagram showing an example of a display image displayed by processing of the endoscope system according to the first embodiment;

FIG. 8 is a flowchart for explaining an example of processing performed in the endoscope system according to the first embodiment;

FIG. 9 is a diagram schematically showing an example of a state of an intestinal tract in a case in which operation of an insertion section is performed according to the processing shown in FIG. 8;

FIG. 10 is a diagram schematically showing an example of a state of the intestinal tract in the case in which the operation of the insertion section is performed according to the processing shown in FIG. 8;

FIG. 11 is a diagram schematically showing an example of a state of the intestinal tract in the case in which the operation of the insertion section is performed according to the processing shown in FIG. 8;

FIG. 12 is a diagram showing an example of a display image displayed by the processing of the endoscope system according to the first embodiment;

FIG. 13 is a diagram for explaining an example of table data used for the processing performed in the endoscope system according to the first embodiment;

FIG. 14 is a flowchart for explaining an example different from the example shown in FIG. 6 of the processing performed in the endoscope system according to the first embodiment;

FIG. 15 is a block diagram for explaining an example of a configuration of an endoscope system according to a second embodiment;

FIG. 16 is a block diagram for explaining an example of a configuration of an examination-situation-information acquiring unit according to the second embodiment;

FIG. 17 is a block diagram for explaining an example of a configuration of a procedure evaluating unit according to the second embodiment;

FIG. 18 is a flowchart for explaining an example of processing performed in the endoscope system according to the second embodiment;

FIG. 19 is a block diagram for explaining an example of a configuration of an endoscope system according to a third embodiment;

FIG. 20 is a block diagram for explaining an example of a configuration of an examination-situation-information acquiring unit according to the third embodiment;

FIG. 21 is a block diagram for explaining an example of a configuration of a procedure evaluating unit according to the third embodiment; and

FIG. 22 is a flowchart for explaining an example of processing performed in the endoscope system according to the third embodiment.

### DETAILED DESCRIPTION OF THE PREFERRED EMBODIMENTS

Embodiments of the present invention are explained below with reference to the drawings.

### First Embodiment

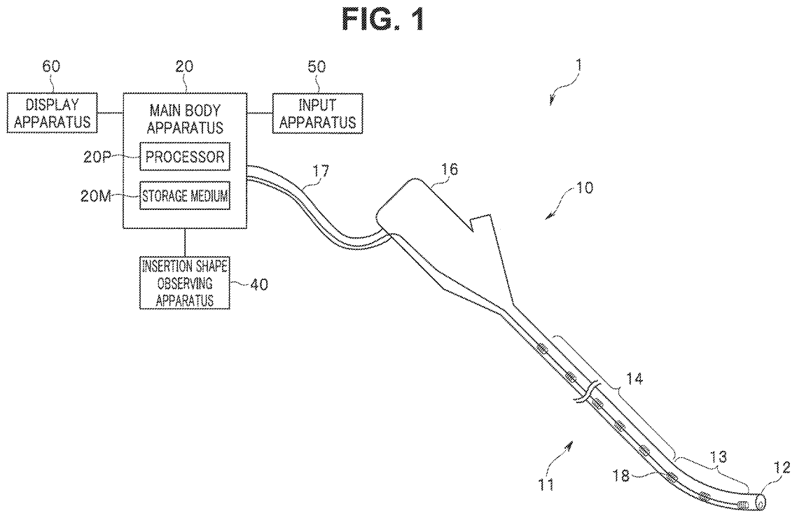

FIG. 1 to FIG. 14 relate to a first embodiment of the present invention.

An endoscope system 1 includes, for example, as shown in FIG. 1, an endoscope 10, a main body apparatus 20, an insertion shape observing apparatus 40, an input apparatus 50, and a display apparatus 60. FIG. 1 is a diagram showing a configuration of a main part of an endoscope system including an endoscopic examination supporting apparatus according to an embodiment.

The endoscope 10 includes an insertion section 11 inserted into a subject, an operation section 16 provided on a proximal end side of the insertion section 11, and a universal cord 17 extended from the operation section 16. The endoscope 10 is removably connected to the main body apparatus 20 via a scope connector (not shown) provided at an end portion of the universal cord 17. Light guides (not shown) for transmitting illumination light supplied from the main body apparatus 20 are provided inside the insertion section 11, the operation section 16, and the universal cord 17.

The insertion section 11 has flexibility and an elongated shape. The insertion section 11 is configured by providing, in order from a distal end side, a hard distal end portion 12, a bendably formed bending section 13, and a long flexible tube section 14 having flexibility. A plurality of source coils 18 that generate a magnetic field corresponding to a coil driving signal supplied from the main body apparatus 20 are provided on insides of the distal end portion 12, the bending section 13, and the flexible tube section 14 at a predetermined interval in a longitudinal direction of the insertion section 11. A treatment instrument channel (not shown) having a shape capable of allowing an elongated treatment instrument to be inserted through and formed to connect a treatment instrument insertion port (not shown) provided in the operation section 16 and a treatment instrument projection port (not shown) provided at the distal end portion 12 is provided on an inside of the insertion section 11. In other words, the endoscope 10 is configured such that the elongated treatment instrument inserted from the treatment instrument insertion port can be inserted through an inside of the treatment instrument channel and a distal end portion of the treatment instrument can be projected from the treatment instrument projection port.

An illumination window (not shown) for emitting, to an object, the illumination light transmitted by the light guide provided inside the insertion section 11 is provided at the distal end portion 12. An image pickup unit 110 (not shown in FIG. 1) configured to perform an operation corresponding to an image pickup control signal supplied from the main body apparatus 20 and pick up an image of the object illuminated by the illumination light emitted through the illumination window and output an image pickup signal is provided at the distal end portion 12. The image pickup unit 110 includes, for example, an observation window on which return light from the object illuminated by the illumination light is made incident and an image sensor such as a color CCD that picks up an image of the return light and outputs an image pickup signal.

The bending section 13 is configured to be able to bend according to operation of an angle knob (not shown) provided in the operation section 16.

The operation section 16 has a shape for enabling a user such as a surgeon to grip and operate the operation section 16. An angle knob configured to be able to perform operation for bending the bending section 13 in up, down, left, and right four directions crossing a longitudinal axis of the insertion section 11 is provided in the operation section 16. One or more scope switches (not shown) capable of carrying out instructions corresponding to input operations of the user are provided in the operation section 16.

The main body apparatus 20 includes one or more processors 20P and a non-transitory storage medium 20M. The main body apparatus 20 includes a function of the endoscopic examination supporting apparatus. The main body apparatus 20 is removably connected to the endoscope 10 via the universal cord 17. The main body apparatus 20 is removably connected to respective units of the insertion shape observing apparatus 40, the input apparatus 50, and the display apparatus 60. The main body apparatus 20 is configured to perform an operation corresponding to an instruction from the input apparatus 50. The main body apparatus 20 is configured to generate an endoscopic image based on an image pickup signal outputted from the endoscope 10 and perform an operation for causing the display apparatus 60 to display the generated endoscopic image. The main body apparatus 20 is configured to be able to generate operation support information including guide information for supporting the inserting operation for the insertion section 11 by the user and to be able to perform an operation for causing the display apparatus 60 to display the generated operation support information. The main body apparatus 20 is configured to be able to generate procedure evaluation information including information obtained by evaluating a procedure including the inserting operation for the insertion section 11 performed by the user during an endoscopic examination and to be able to perform an operation for causing the display apparatus 60 to display the generated procedure evaluation information.

The insertion shape observing apparatus 40 is configured to detect magnetic fields emitted from the respective source coils 18 provided in the insertion section 11 and acquire positions of the respective plurality of source coils 18 based on intensity of the detected magnetic fields. The insertion shape observing apparatus 40 is configured to generate insertion position information indicating the positions of the respective plurality of source coils 18 acquired as explained above and output the insertion position information to the main body apparatus 20. In other words, the insertion shape observing apparatus 40 is configured to detect an insertion position of the insertion section inserted into the subject and acquire insertion position information and output the acquired insertion position information to the main body apparatus 20.

The input apparatus 50 includes one or more input interfaces operated by the user such as a mouse, a keyboard, and a touch panel. The input apparatus 50 is configured to be able to output an instruction corresponding to operation of the user to the main body apparatus 20.

The display apparatus 60 includes, for example, a liquid crystal monitor. The display apparatus 60 is configured to be able to display, on a screen, an endoscopic image and the like outputted from the main body apparatus 20.

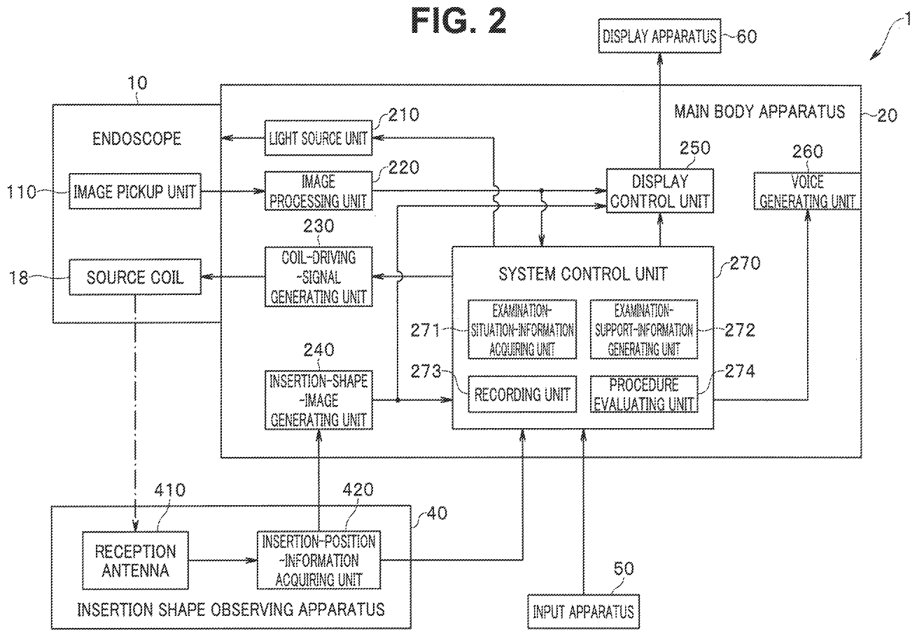

The main body apparatus 20 includes, as shown in FIG. 2, a light source unit 210, an image processing unit 220, a coil-driving-signal generating unit 230, an insertion-shape-image generating unit 240, a display control unit 250, a voice generating unit 260, and a system control unit 270. FIG. 2 is a block diagram for explaining an example of a configuration of an endoscope system according to the first embodiment.

The light source unit 210 includes, for example, one or more LEDs or one or more lamps as light sources. The light source unit 210 is configured to be able to generate illumination light for illuminating an inside of the subject into which the insertion section 11 is inserted and supply the illumination light to the endoscope 10. The light source unit 210 is configured to be able to change a light amount of the illumination light according to a system control signal supplied from the system control unit 270.

The image processing unit 220 includes, for example, an image processing circuit. The image processing unit 220 is configured to apply predetermined processing to an image pickup signal outputted from the endoscope 10 to thereby generate an endoscopic image and sequentially output the generated endoscopic image to the display control unit 250 and the system control unit 270 frame by frame.

The coil-driving-signal generating unit 230 includes, for example, a drive circuit. The coil-driving-signal generating unit 230 is configured to generate, according to the system control signal supplied from the system control unit 270, a coil driving signal for driving the source coils 18 and output the coil driving signal.

The insertion-shape-image generating unit 240 is configured to generate, based on the insertion position information outputted from the insertion shape observing apparatus 40, an insertion shape image obtained by two-dimensionally visualizing the insertion shape of the insertion section 11 inserted into the subject. The insertion-shape-image generating unit 240 is configured to output the insertion shape image generated as explained above to the display control unit 250 and the system control unit 270.

The display control unit 250 is configured to perform processing for generating a display image including the endoscopic image outputted from the image processing unit 220 and perform processing for causing the display apparatus 60 to display the generated display image. The display control unit 250 is configured to be able to perform processing for causing the display apparatus 60 to display the insertion shape image outputted from the insertion-shape-image generating unit 240. The display control unit 250 is configured to be able to perform processing for generating visual information such as a character string and a sign corresponding to the operation support information outputted from the system control unit 270 and perform processing for causing the display apparatus 60 to display the display image including the generated visual information. The display control unit 250 is configured to be able to perform processing for generating visual information such as a character string and a sign corresponding to the procedure evaluation information (explained below) outputted from the system control unit 270 and perform processing for causing the display apparatus 60 to display the display image including the generated visual information.

The voice generating unit 260 includes, for example, a speaker. The voice generating unit 260 is configured to generate voice corresponding to the guide information included in the operation support information outputted from the system control unit 270 and perform an operation for outputting the generated voice to an outside of the main body apparatus 20.

The system control unit 270 is configured to generate and output a system control signal for causing the endoscope system 1 to perform an operation corresponding to instructions and the like from the operation section 16 and the input apparatus 50. The system control unit 270 is configured to generate operation support information based on at least one piece of information among the endoscopic image outputted from the image processing unit 220, the insertion shape information outputted from the insertion-shape-image generating unit 240, and the insertion position information outputted from the insertion shape observing apparatus 40 and output the generated operation support information to the display control unit 250 and the voice generating unit 260. The system control unit 270 is configured to generate procedure evaluation information based on endoscopic examination information (explained below) recorded during the endoscopic examination and output the generated procedure evaluation information to the display control unit 250. The system control unit 270 includes an examination-situation-information acquiring unit 271, an examination-support-information generating unit 272, a recording unit 273, and a procedure evaluating unit 274.

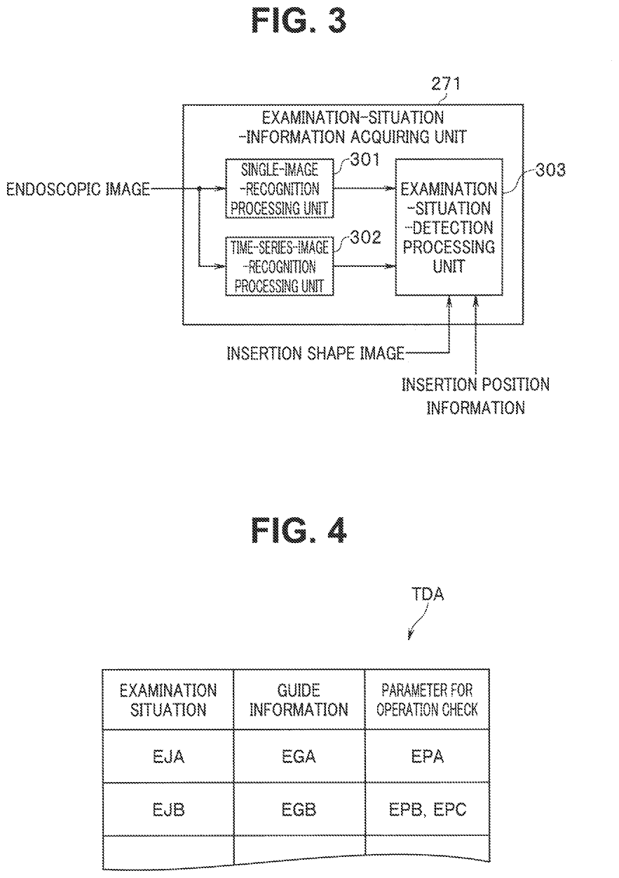

The examination-situation-information acquiring unit 271 is configured to perform, based on the endoscopic image outputted from the image processing unit 220, the insertion shape image outputted from the insertion-shape-image generating unit 240, and the insertion position information outputted from the insertion shape observing apparatus 40, processing for acquiring examination situation information equivalent to information indicating a present examination situation in an examination (an endoscopic examination) performed on the subject using the endoscope 10. More specifically, the examination-situation-information acquiring unit 271 includes, for example, as shown in FIG. 3, a single-image-recognition processing unit 301, a time-series-image-recognition processing unit 302, and an examination-situation-detection processing unit 303. FIG. 3 is a block diagram for explaining an example of a configuration of an examination-situation-information acquiring unit according to the first embodiment.

The single-image-recognition processing unit 301 includes a recognizer CLP created by implementing an algorithm for object detection such as YOLO in a predetermined neural network such as Darknet and performing learning. Alternatively, the single-image-recognition processing unit 301 includes a recognizer CLP created by learning, with a learning method such as deep learning, respective coupling coefficients (weights) in a multilayer neural network including, for example, an input layer, one or more convolutional layers, and an output layer. The single-image-recognition processing unit 301 is configured to perform processing using the recognizer CLP created as explained above to thereby obtain a recognition result obtained by recognizing, as one situation among a predetermined plurality of situations, an examination situation corresponding to an endoscopic image for one frame generated by the image processing unit 220.

At the creation time of the recognizer CLP explained above, machine learning using teacher data including, for example, an endoscopic image for one frame obtained by picking up an image of an inside of an intestinal tract and a label indicating a classification result obtained by classifying an examination situation corresponding to the endoscopic image for the one frame into one situation among the predetermined plurality of situations.

Accordingly, with the recognizer CLP explained above, for example, by acquiring multidimensional data such as pixel values of respective pixels included in the endoscopic image for the one frame generated by the image processing unit 220 and inputting the multidimensional data to an input layer of a neural network as input data, it is possible to acquire, as output data outputted from an output layer of the neural network, a plurality of likelihoods corresponding to the respective situations that can be classified as examination situations corresponding to the endoscopic image for the one frame. With the processing using the recognizer CLP explained above, for example, it is possible to obtain, as a recognition result of an examination situation corresponding to the endoscopic image for the one frame generated by the image processing unit 220, one situation corresponding to highest one likelihood among the plurality of likelihoods included in the output data outputted from the output layer of the neural network.

The single-image-recognition processing unit 301 is configured to be able to obtain, by performing the processing using the recognizer CLP explained above, as information included in the recognition result of the examination situation corresponding to the endoscopic image for the one frame generated by the image processing unit 220, for example, at least one piece of information among presence or absence of a lumen in a visual field range of the image pickup unit 110 provided in the insertion section 11 inserted into the intestinal tract, a type of a lumen present in the visual field range, an anatomical site corresponding to the visual field range in the intestinal tract, a position of the lumen present in the visual field range, a direction of the lumen present in the visual field range, presence or absence of implementation of a narrowband optical observation in the intestinal tract, presence or absence of implementation of an enlarged observation in the intestinal tract, presence or absence of spraying of a pigment (indigo carmine or the like) in the intestinal tract, presence or absence of use of a treatment instrument in the intestinal tract, presence or absence of sampling of a tissue in the intestinal tract, presence or absence of a residue in the visual field range, presence of absence of a diverticulum in the visual field range, and presence or absence of a lesion in the visual field range.

In the present embodiment, with the processing of the single-image-recognition processing unit 301, when an entire region or substantially the entire region of the endoscopic image is red because, for example, the distal end portion 12 is excessively close to an internal wall of the intestinal tract, it is possible to obtain a recognition result indicating that a lumen is absent in the visual field range of the image pickup unit 110 provided in the insertion section 11 inserted into the intestinal tract. In the present embodiment, as a recognition result of the type of the lumen explained above, for example, it is possible to obtain a recognition result capable of specifying to which of an opened lumen equivalent to a lumen in which a moving destination of the distal end portion 12 is not closed and that is present in an easily visually recognizable position, a closed lumen equivalent to a lumen in which a moving destination of the distal end portion 12 is closed, and a folded lumen equivalent to a lumen in which a moving destination of the distal end portion 12 is not closed and that is present in a hardly recognizable position, the lumen being present in the visual field range of the image pickup unit 110 provided in the insertion section 11 corresponds.

The time-series-image-recognition processing unit 302 includes, for example, a recognizer CLQ created by learning a 3D-CNN (a three-dimensional convolutional neural network) with a learning method such as deep learning. The time-series-image-recognition processing unit 302 is configured to perform processing using the recognizer CLQ created as explained above to thereby obtain a recognition result obtained by recognizing, as one situation among a predetermined plurality of situations, an examination situation corresponding to an endoscopic image for a temporally continuous plurality of frames generated by the image processing unit 220.

At the creation time of the recognizer CLQ explained above, for example, machine learning using teacher data including endoscopic images for a plurality of frames obtained by continuously picking up images of the inside of the intestinal tract and a label indicating a classification result obtained by classifying an examination situation corresponding to the endoscopic images for the plurality of frames into one situation among the predetermined plurality of situations is performed.

Accordingly, with the recognizer CLQ explained above, for example, by acquiring multidimensional data such as pixel values of respective pixels included in the endoscopic images for the temporally continuous plurality of frames generated by the image processing unit 220 and inputting the multidimensional data to an input layer of a neural network as input data, it is possible to acquire, as output data outputted from an output layer of the neural network, a plurality of likelihoods corresponding to the respective situations that can be classified as examination situations corresponding to the endoscopic images for the plurality of frames. With the processing using the recognizer CLQ explained above, for example, it is possible to obtain, as a recognition result of an examination situation corresponding to the temporally continuous endoscopic images for the plurality of frames generated by the image processing unit 220, one situation corresponding to highest one likelihood among the plurality of likelihoods included in the output data outputted from the output layer of the neural network.

The time-series-image-recognition processing unit 302 is configured to be able to, by performing the processing using the recognizer CLQ explained above, obtain, as information included in a recognition result of an examination situation corresponding to the endoscopic images for the temporally continuous plurality of frames generated by the image processing unit 220, at least one piece of information among, for example, a change in a distance with respect to a lumen present in the visual field range of the image pickup unit 110 provided in the insertion section 11 inserted into the intestinal tract, a change in a distance with respect to a wrinkle present in the visual field range, presence or absence of water feeding in the visual field range, presence or absence of air feeding in the visual field range, and presence or absence of suction in the visual field range.

Note that, in the present embodiment, with the processing of the time-series-image-recognition processing unit 302, for example, when the internal wall of the intestinal tract approaches in a state in which the distal end portion 12 is not displaced or substantially not displaced, it is possible to obtain a recognition result indicating that suction is performed in the visual field range of the image pickup unit 110 provided in the insertion section 11 inserted into the intestinal tract.

The examination-situation-detection processing unit 303 is configured to perform processing using at least one of the insertion position information outputted from the insertion shape observing apparatus 40, the recognition result obtained by the single-image-recognition processing unit 301, the recognition result obtained by the time-series-image-recognition processing unit 302, or the insertion shape image outputted from the insertion-shape-image generating unit 240 to thereby acquire examination situation information indicating a present examination situation in the examination (the endoscopic examination) performed on the subject using the endoscope 10.

Note that, in the present embodiment, explanation is performed assuming that the examination-situation-detection processing unit 303 calculates each of a present insertion length of the insertion section 11 and a present bending angle of the bending section 13 based on at least one of the insertion position information outputted from the insertion shape observing apparatus 40 or the insertion shape image outputted from the insertion-shape-image generating unit 240. In the present embodiment, explanation is performed assuming that the examination-situation-detection processing unit 303 performs substantially the same processing as analysis processing of the insertion-shape-analysis processing unit 312 explained below based on, for example, at least one of the insertion position information outputted from the insertion shape observing apparatus 40 or the insertion shape image outputted from the insertion-shape-image generating unit 240 to thereby calculate an estimated pressing force estimated to be currently applied to an intestinal wall by the insertion section 11 inserted into the intestinal tract. In the present embodiment, explanation is performed assuming that examination situation information including information indicating present operation amounts in one or more parameters relating to inserting operation for the insertion section 11 such as a rotation angle in a case in which the insertion section 11 is rotated around an insertion axis (a longitudinal axis) is acquired by the processing of the examination-situation-detection processing unit 303.

The examination-support-information generating unit 272 is configured to, based on the examination situation information acquired by the examination-situation-information acquiring unit 271, perform processing for generating operation support information including guide information for supporting the inserting operation for the insertion section 11 performed by the user who operates the endoscope 10 in the endoscopic examination and perform processing for outputting the generated operation support information to the display control unit 250 and the voice generating unit 260.

More specifically, the examination-support-information generating unit 272 is configured to, for example, read table data TDA shown in FIG. 4 from the storage medium 20M, select, out of a plurality of pieces of guide information included in the read table data TDA, one guide information corresponding to a present examination situation specified by the examination situation information acquired by the examination-situation-information acquiring unit 271, and generate operation support information including the selected one guide information and output the operation support information to the display control unit 250 and the voice generating unit 260. FIG. 4 is a diagram for explaining an example of table data used for processing performed in the endoscope system according to the first embodiment.

An “examination situation” field of the table data TDA shown in FIG. 4 includes a plurality of items indicating overviews (for example, “N loop formation” and “pressing force excessive”) of respective examination situations that can be detected by the processing of the examination-situation-detection processing unit 303. A “guide information” field of the table data TDA shown in FIG. 4 includes a plurality of items indicating, for example, types of guides (for example, “N loop release” and “pressing force decrease”) corresponding to the respective items of the “examination situation” field and operation methods (for example, “please press” and “please twist to the right”) corresponding to the types of the guides. A “parameter for operation check” field of the table data TDA shown in FIG. 4 includes a plurality of items in which one or more parameters used to determine whether kinds of operation corresponding to the respective items of the “guide information” field end are set.

Accordingly, for example, when the present examination situation specified based on the examination situation information acquired by the examination-situation-information acquiring unit 271 is an examination situation EJA, the examination-support-information generating unit 272 selects guide information EGA corresponding to the examination situation EJA and generates operation support information including the guide information EGA and outputs the operation support information to the display control unit 250 and the voice generating unit 260. For example, when the present examination situation specified based on the examination situation information acquired by the examination-situation-information acquiring unit 271 is an examination situation EJB, the examination-support-information generating unit 272 selects guide information EGB corresponding to the examination situation EJB and generates operation support information including the guide information EGB and outputs the operation support information to the display control unit 250 and the voice generating unit 260.

The examination-support-information generating unit 272 is configured to perform, based on the examination situation information acquired by the examination-situation-information acquiring unit 271, for example, every time an endoscopic image for one frame is outputted from the image processing unit 220, processing for determining whether it is necessary to update the operation support information currently being outputted.

More specifically, for example, when selecting the guide information EGA out of each piece of guide information included in the table data TDA shown in FIG. 4, the examination-support-information generating unit 272 acquires a parameter EPA as a parameter for operation check corresponding to the guide information EGA and performs processing such as threshold determination using the acquired parameter EPA and the examination situation information acquired by the examination-situation-information acquiring unit 271 to thereby determine whether it is necessary to update the operation support information including the guide information EGA currently being outputted. For example, when selecting the guide information EGB out of each piece of guide information included in the table data TDA shown in FIG. 4, the examination-support-information generating unit 272 acquires parameters EPB and EPC as parameters for operation check corresponding to the guide information EGB and performs processing such as threshold determination using the acquired parameters EPB and EPC and the examination situation information acquired by the examination-situation-information acquiring unit 271 to thereby determine whether it is necessary to update the operation support information including the guide information EGB currently being outputted.

The examination-support-information generating unit 272 is configured to, when obtaining a determination result that it is necessary to update the operation support information currently being outputted, based on the examination situation information acquired by the examination-situation-information acquiring unit 271, perform processing for generating new operation support information including guide information different from the guide information included in the operation support information and perform processing for outputting the generated new operation support information to the display control unit 250 and the voice generating unit 260. The examination-support-information generating unit 272 is configured to, when obtaining a determination result that it is unnecessary to update the operation support information being currently outputted, perform processing for outputting the operation support information to the display control unit 250 and the voice generating unit 260 again.

The recording unit 273 is configured to perform an operation for recording, as endoscopic examination information, an image, information, and the like obtained during the examination (the endoscopic examination) performed on the subject using the endoscope 10.

More specifically, the recording unit 273 performs an operation for respectively recording, during the endoscopic examination, for example, the endoscopic image outputted from the image processing unit 220, the insertion position information outputted from the insertion shape observing apparatus 40, and the insertion shape image outputted from the insertion-shape-image generating unit 240. Every time one piece of operation support information is generated by the examination-support-information generating unit 272, the recording unit 273 performs an operation for recording, as operation support time information, information indicating a time in which the one piece of operation support information is continuously outputted to the display control unit 250.

In other words, the recording unit 273 records, as endoscopic examination information in one endoscopic examination, information correlating an endoscopic image group including endoscopic images for a plurality of frames sequentially outputted from the image processing unit 220 during the one endoscopic examination (obtained during the one endoscopic examination), an insertion position information group including a plurality of insertion position information sequentially outputted from the insertion shape observing apparatus 40 during the one endoscopic examination (obtained during the one endoscopic examination), an insertion shape image group including a plurality of insertion shape images sequentially outputted from the insertion-shape-image generating unit 240 during the one endoscopic examination, and an operation support time information group indicating output times and output contents of a respective plurality of pieces of operation support information outputted from the examination-support-information generating unit 272 to the display control unit 250 during the one endoscopic examination. In other words, the operation support time information group recorded by the recording unit 273 includes information capable of specifying, for each piece of guide information, a presentation time in which a plurality of pieces of guide information included in a plurality of pieces of operation support information generated during the one endoscopic examination are presented to the user.

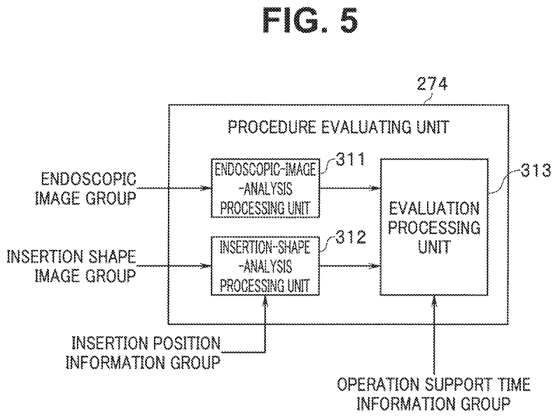

The procedure evaluating unit 274 is configured to, based on the endoscopic examination information recorded by the recording unit 273, generate procedure evaluation information including information obtained by evaluating a procedure including inserting operation for the insertion section 11 performed by the user during the examination (the endoscopic examination) performed on the subject using the endoscope 10 and perform processing for outputting the generated procedure evaluation information to the display control unit 250. More specifically, the procedure evaluating unit 274 includes, for example, as shown in FIG. 5, an endoscopic-image-analysis processing unit 311, an insertion-shape-analysis processing unit 312, and an evaluation processing unit 313. FIG. 5 is a block diagram for explaining an example of a configuration of a procedure evaluating unit according to the first embodiment.

The endoscopic-image-analysis processing unit 311 is configured to perform analysis processing based on the endoscopic image group included in the endoscopic examination information recorded by the recording unit 273 to thereby obtain an analysis result including analysis values of one or more evaluation indicators corresponding to operation and the like estimated to be performed by the user during the endoscopic examination.

More specifically, the endoscopic-image-analysis processing unit 311 is configured to perform the analysis processing based on the endoscopic image group included in the endoscopic examination information recorded by the recording unit 273 to thereby obtain an analysis result including an analysis value of at least one evaluation indicator among, for example, presence or absence of a lumen in an intestinal tract set as an examination target of the endoscopic examination, a type of the lumen in the intestinal tract, a position of the lumen in the intestinal tract, presence or absence of a residue in the intestinal tract, a position of the residue in the intestinal tract, a ratio of an image including the residue in the endoscopic image group, the number of times of water feeding in the intestinal tract, the number of times of suction in the intestinal tract, presence or absence of a lesion in the intestinal tract, the number of times of sampling of a tissue in the intestinal tract, a distance between the tissue set as a sampling target in the intestinal tract and the distal end portion 12, the number of times of spraying of a pigment (indigo carmine or the like) in the intestinal tract, the number of times of use of an enlarged observation in the intestinal tract, a use time of the enlarged observation in the intestinal tract, the number of times of use of a narrowband optical observation in the intestinal tract, a use time of the narrowband optical observation in the intestinal tract, and a rotation amount of the insertion section 11 inserted into the intestinal tract.

Note that, in the present embodiment, it is assumed that, as an analysis value of an evaluation indicator corresponding to the presence or absence of a lumen in the intestinal tract, for example, when an examination time equivalent to a time required for one endoscopic examination calculated based on a total number of frames of the endoscopic image group included in the endoscopic examination information recorded by the recording unit 273 is represented as PEA, information capable of specifying each of a ratio of a time PLY, in which any lumen present in the intestinal tract is within a visual field range of the image pickup unit 110, to the examination time PEA and a ratio of a time PLN, in which the lumen present in the intestinal tract is not within the visual field range, to the examination time PEA is obtained. In the present embodiment, it is assumed that, as an analysis value of an evaluation indicator corresponding to the type of the lumen in the intestinal tract, for example, information capable of specifying each of a ratio of a time PLA, in which an opened lumen is within the visual field range of the image pickup unit 110, to the time PLY, a ratio of a time PLB, in which a closed lumen is within the visual field range, to the time PLY, and a ratio of a time PLC, in which a folded lumen is within the visual field range, to the time PLY is obtained. In the present embodiment, it is assumed that, as an analysis value of an evaluation indicator corresponding to the position of the lumen in the intestinal tract, for example, information capable of specifying coordinate positions in images of the lumen included in respective endoscopic images obtained in the time PLY is obtained. In the present embodiment, it is assumed that, as an analysis value of an evaluation indicator corresponding to the presence or absence of the residue in the intestinal tract, for example, information capable of specifying each of a ratio of a time PRY, in which the residue present in the intestinal tract is within the visual field range of the image pickup unit 110, to the examination time PEA and a ratio of a time PRN, in which the residue present in the intestinal tract is not within the visual field range to the examination time PEA, is obtained. In the present embodiment, it is assumed that, as an analysis value of an evaluation indicator corresponding to the position of the residue in the intestinal tract, for example, information capable of specifying positions in images of the residue included in respective endoscopic images obtained in the time PRY is obtained. In the present embodiment, it is assumed that, as an analysis value of an evaluation indicator corresponding to the rotation amount of the insertion section 11 inserted into the intestinal tract, information indicating a rotation angle in a case in which the insertion section 11 is rotated around the insertion axis (the longitudinal axis) is obtained.

The insertion-shape-analysis processing unit 312 is configured to perform analysis processing based on the insertion shape image group and the insertion position information group included in the endoscopic examination information recorded by the recording unit 273 to thereby obtain an analysis result including analysis values of one or more evaluation indicators corresponding to operation and the like estimated as being performed by the user during the endoscopic examination.

More specifically, the insertion-shape-analysis processing unit 312 is configured to perform the analysis processing based on the insertion shape image group and the insertion position information group included in the endoscopic examination information recorded by the recording unit 273 to thereby obtain an analysis result including an analysis value of at least one evaluation indicator among, for example, estimated stay times in respective anatomical sites in the intestinal tract set as the examination target of the endoscopic examination, an insertion length of the insertion section 11 inserted into the intestinal tract, inserting speed in inserting the insertion section 11 into the intestinal tract, inserting acceleration in inserting the insertion section 11 into the intestinal tract, inserting jerk in inserting the insertion section 11 into the intestinal tract, removing speed in removing the insertion section 11 from the intestinal tract, removing acceleration in removing the insertion section 11 from the intestinal tract, removing jerk in removing the insertion section 11 from the intestinal tract, speed of the distal end portion 12 inserted into the intestinal tract, acceleration of the distal end portion 12 inserted into the intestinal tract, jerk of the distal end portion 12 inserted into the intestinal tract, a bending angle of the bending section 13 inserted into the intestinal tract, bending angular velocity of the bending section 13 inserted into the intestinal tract, bending angular acceleration of the bending section 13 inserted into the intestinal tract, bending angular jerk of the bending section 13 inserted into the intestinal tract, a rotation amount of the insertion section 11 inserted into the intestinal tract, a path length of the insertion section 11 inserted into the intestinal tract, an estimated pressing force estimated to be applied to the intestinal wall by the insertion section 11 inserted into the intestinal tract, the number of times buckling occurs in the insertion section 11 inserted into the intestinal tract, a time in which the buckling continues to occur in the insertion section 11 inserted into the intestinal tract, the number of times the insertion section 11 inserted into the intestinal tract forms a stick shape, a time in which the insertion section 11 inserted into the intestinal tract continues to form the stick shape, the number of times the insertion section 11 inserted into the intestinal tract forms a loop shape, a time in which the insertion section 11 inserted into the intestinal tract continues to form the loop shape, a size of the loop shape formed by the insertion section 11 inserted into the intestinal tract, and a rotation amount of the insertion section 11 inserted into the intestinal tract.

Note that, in the present embodiment, it is assumed that, as analysis values of evaluation indicators corresponding to the estimated stay times in the respective anatomical sites in the intestinal tract, for example, information indicating a stay time of the distal end portion 12 for each of the sites in the intestinal tract estimated based on at least one of the insertion shape image group or the insertion position information group included in the endoscopic examination information recorded by the recording unit 273 and an insertion length of the insertion section 11 inserted into the intestinal tract is obtained. In the present embodiment, it is assumed that, as an analysis value of an evaluation indicator corresponding to the path length of the insertion section 11 inserted into the intestinal tract, for example, information indicating a total value of movement amounts accumulated according to respective kinds of operation for displacing the distal end portion 12 such as insertion, removal, and bending of the insertion section 11 is obtained. In the present embodiment, it is assumed that, as an analysis value of an evaluation indicator corresponding to the estimated pressing force estimated as being applied to the intestinal wall by the insertion section 11 inserted into the intestinal tract, for example, information indicating magnitude of a force estimated based on one insertion shape specified from at least one of the insertion shape image group or the insertion position information group included in the endoscopic examination information recorded by the recording unit 273 and an estimated value of bending rigidity estimated from the one insertion shape is obtained. In the present embodiment, it is assumed that, as an analysis value of an evaluation indicator corresponding to the size of the loop shape formed by the insertion section 11 inserted into the intestinal tract, for example, information indicating length in a vertical direction of a portion corresponding to the loop shape (equivalent to an up-down direction in a human body) and information indicating length in a lateral direction of the portion corresponding to the loop shape (equivalent to a left-right direction in the human body) are respectively obtained.

The evaluation processing unit 313 is configured to perform processing using the analysis result obtained by the endoscopic-image-analysis processing unit 311, the analysis result obtained by the insertion-shape-analysis processing unit 312, and the operation support time information group included in the endoscopic examination information recorded by the recording unit 273 to thereby generate procedure evaluation information and perform processing for outputting the generated procedure evaluation information to the display control unit 250.

In the present embodiment, at least a part of functions of the main body apparatus 20 only has to be realized by the processor 20P. In the present embodiment, at least a part of the main body apparatus 20 may be configured as individual electronic circuits or may be configured as a circuit block in an integrated circuit such as an FPGA (field programmable gate array). By modifying the configuration according to the present embodiment as appropriate, for example, a computer may read, from the storage medium 20M such as a memory, a program for executing at least a part of the functions of the main body apparatus 20 and perform an operation corresponding to the read program.

The insertion shape observing apparatus 40 includes, as shown in FIG. 2, a reception antenna 410 and an insertion-position-information acquiring unit 420.

The reception antenna 410 includes, for example, a plurality of coils for three-dimensionally detecting magnetic fields emitted from the respective plurality of source coils 18. The reception antenna 410 is configured to detect the magnetic fields emitted from the respective plurality of source coils 18 and generate a magnetic field detection signal corresponding to intensity of the detected magnetic fields and output the magnetic field detection signal to the insertion-position-information acquiring unit 420.

The insertion-position-information acquiring unit 420 is configured to acquire positions of the respective plurality of source coils 18 based on the magnetic field detection signal outputted from the reception antenna 410. The insertion-position-information acquiring unit 420 is configured to generate insertion position information indicating the positions of the respective plurality of source coils 18 acquired as explained above and output the insertion position information to the insertion-shape-image generating unit 240 and the system control unit 270.

More specifically, the insertion-position-information acquiring unit 420 acquires, as the positions of the respective plurality of source coils 18, for example, a plurality of three-dimensional coordinate values in a space coordinate system virtually set such that a predetermined position (an anus or the like) of the subject into which the insertion section 11 is inserted is an origin or a reference point. The insertion-position-information acquiring unit 420 generates insertion position information including the plurality of three-dimensional coordinate values acquired as explained above and outputs the insertion position information to the insertion-shape-image generating unit 240 and the system control unit 270. In such a case, the insertion-shape-image generating unit 240 performs, for example, processing for acquiring a plurality of two-dimensional coordinate values corresponding to the respective plurality of three-dimensional coordinate values included in the insertion position information outputted from the insertion-position-information acquiring unit 420, processing for interpolating the acquired plurality of two-dimensional coordinate values, and processing for generating an insertion shape image corresponding to the interpolated plurality of two-dimensional coordinate values.

In other words, the insertion shape information indicating the insertion shape of the insertion section 11 inserted into the subject includes insertion position information indicating an insertion position of the insertion section 11 inserted into the subject and an insertion shape image obtained by visualizing an insertion shape of the insertion section 11 inserted into the subject. The insertion shape information group recorded by the recording unit 273 in the one endoscopic examination includes an insertion position information group including a plurality of pieces of insertion position information sequentially outputted from the insertion shape observing apparatus 40 during the one endoscopic examination and an insertion shape image group including a plurality of insertion shape images sequentially outputted from the insertion-shape-image generating unit 240 during the one endoscopic examination.

In the present embodiment, at least a part of the insertion shape observing apparatus 40 may be configured as an electronic circuit or may be configured as a circuit block in an integrated circuit such as an FPGA (field programmable gate array). In the present embodiment, for example, the insertion shape observing apparatus 40 may include one or more processors (CPUs or the like).

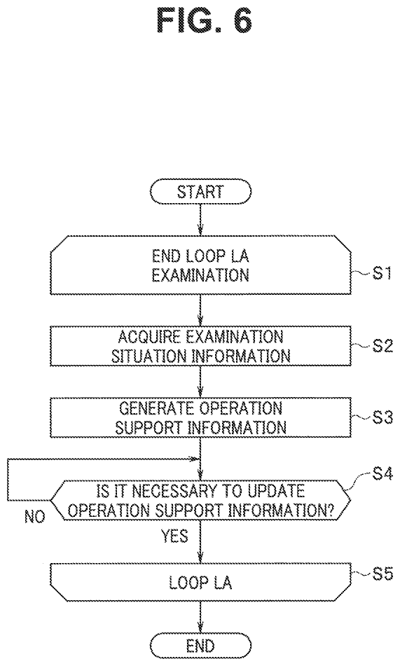

Subsequently, action of the present embodiment is explained with reference to FIG. 6 and the like. FIG. 6 is a flowchart for explaining an example of processing performed in the endoscope system according to the first embodiment.

After connecting the respective sections of the endoscope system 1 and turning on the endoscope system 1, the user starts operation for inserting the insertion section 11 from an anus into an inside of a large intestine of the subject.

With the operation of the user explained above, an object is irradiated with illumination light supplied from the light source unit 210, an image of the object irradiated with the illumination light is picked up by the image pickup unit 110, and an endoscopic image obtained by picking up an image of the object is outputted from the image processing unit 220 to the display control unit 250 and the system control unit 270. With the operation of the user explained above, a coil driving signal is supplied from the coil-driving-signal generating unit 230, magnetic fields are emitted from the respective plurality of source coils 18 according to the coil driving signal, insertion position information obtained by detecting the magnetic fields is outputted from the insertion-position-information acquiring unit 420 to the insertion-shape-image generating unit 240 and the system control unit 270, and an insertion shape image generated according to the insertion position information is outputted from the insertion-shape-image generating unit 240 to the system control unit 270.

For example, immediately after starting the insertion of the insertion section 11 into the large intestine of the subject, the user operates an examination start switch (not shown) of the input apparatus 50 to thereby perform an instruction for starting an operation relating to support of the endoscopic examination for the subject.

When detecting the instruction from the examination start switch of the input apparatus 50, the recording unit 273 starts an operation for respectively recording an endoscopic image outputted from the image processing unit 220, insertion position information outputted from the insertion shape observing apparatus 40, an insertion shape image outputted from the insertion-shape-image generating unit 240, and an output time and output content of operation support information outputted from the examination-support-information generating unit 272 to the display control unit 250.

When detecting the instruction from the examination start switch of the input apparatus 50, the examination-situation-information acquiring unit 271 and the examination-support-information generating unit 272 start processing of a loop LA, which is processing for performing operation support corresponding to a present examination situation in the endoscopic examination (step S1 in FIG. 6).

The examination-situation-information acquiring unit 271 performs, based on the endoscopic image outputted from the image processing unit 220, the insertion shape image outputted from the insertion-shape-image generating unit 240, and the insertion position information outputted from the insertion shape observing apparatus 40, processing for acquiring examination situation information indicating the present examination situation in the endoscopic examination (step S2 in FIG. 6).

The examination-support-information generating unit 272, based on the examination situation information acquired by the examination-situation-information acquiring unit 271, performs processing for generating operation support information and performs processing for outputting the generated operation support information to the display control unit 250 and the voice generating unit 260 (step S3 in FIG. 6).

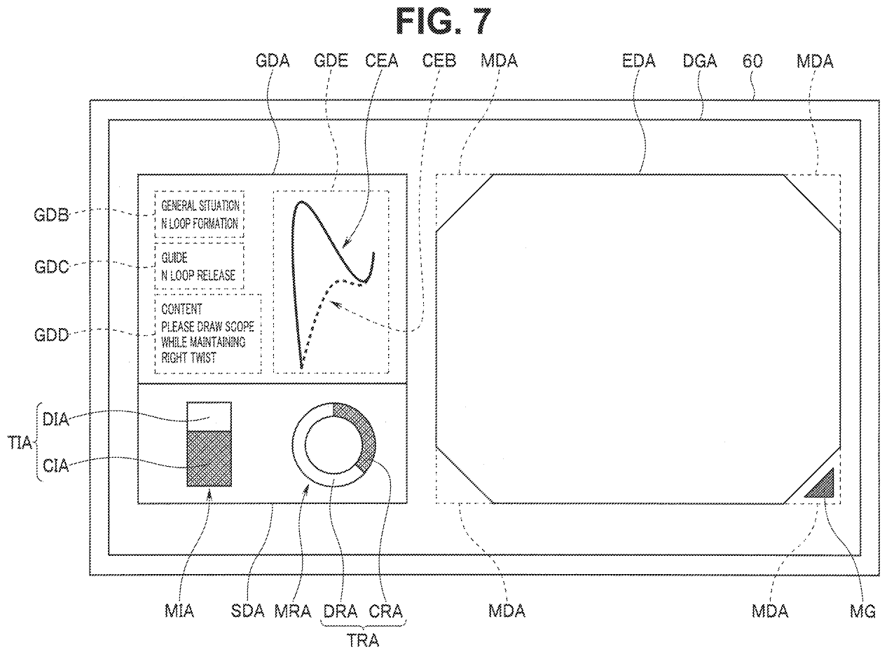

The display control unit 250, based on the endoscopic image outputted from the image processing unit 220 and the operation support information outputted from the system control unit 270, for example performs processing for generating a display image DGA shown in FIG. 7 and performs processing for causing the display apparatus 60 to display the generated display image DGA. FIG. 7 is a diagram showing an example of a display image displayed by processing of the endoscope system according to the first embodiment.

The display image DGA includes an endoscopic image display region EDA, mark display regions MDA, a guide display region GDA, and an operation amount display region SDA.

The endoscopic image display region EDA is set as a region for displaying an endoscopic image EG outputted from the image processing unit 220.

In FIG. 7, for example, the mark display regions MDA are set as four triangular regions located outside the endoscopic image display region EDA and located in four directions on an upper left side, a lower left side, an upper right side, and a lower right side when viewed from a center of the endoscopic image display region EDA (see FIG. 7). Note that the mark display regions MDA may be provided in any positions around the endoscopic image display region EDA having an octagonal shape. The mark display regions MDA are set as regions for displaying a mark MG such as an arrow indicating a direction of a lumen closest from a visual field range of the image pickup unit 110 corresponding to the endoscopic image EG when a lumen is absent in the endoscopic image EG currently displayed in the endoscopic image display region EDA.

The guide display region GDA is set as a region for displaying a character string, a sign, and the like generated based on the operation support information outputted from the system control unit 270. The guide display region GDA includes, for example, as shown in FIG. 7, a general situation display region GDB, a guide type display region GDC, a guide content display region GDD, and an operation example display region GDE.

The general situation display region GDB is set as a region for displaying a character string indicating an overview of a present examination situation in the endoscopic examination. Display content of the general situation display region GDB changes according to guide information included in the operation support information outputted from the system control unit 270.

The guide type display region GDC is set as a region for displaying a character string indicating a type of an operation guide for supporting inserting operation for the insertion section 11 according to the present examination situation in the endoscopic examination. Display content of the guide type display region GDC changes according to the guide information included in the operation support information outputted from the system control unit 270.

The guide content display region GDD is set as a region for displaying a character string indicating a specific operation method for the insertion section 11 corresponding to a type of an operation guide currently displayed in the guide type display region GDC. Display content of the guide content display region GDD changes according to the guide information included in the operation support information outputted from the system control unit 270.

The operation example display region GDE is set as a region for displaying a figure indicating a change in an insertion shape of the insertion section 11 before operation corresponding to a specific operation method currently displayed in the guide content display region GDD and after the operation is performed. More specifically, for example, as shown in FIG. 7, the operation example display region GDE is set as a region capable of displaying a solid curved line CEA indicating an insertion shape of the insertion section 11 before operation relating to release of the N loop is performed and a broken curved line CEB indicating an insertion shape of the insertion section 11 after the operation is performed. Display content of the operation example display region GDE changes according to the guide information included in the operation support information outputted from the system control unit 270.

The operation amount display region SDA is set as a region for displaying a figure or the like indicating a difference amount between a target operation amount necessary for ending operation corresponding to display contents of the guide type display region GDC and the guide content display region GDD and a present operation amount in the operation. More specifically, for example, as shown in FIG. 7, the operation amount display region SDA is set as a region capable of displaying an insertion length meter MIA indicating a difference amount DIA between a target insertion length TIA of the insertion section 11 and a present insertion length CIA of the insertion section 11. For example, as shown in FIG. 7, the operation amount display region SDA is set as a region capable of displaying a rotation amount meter MRA indicating a difference amount DRA between a target rotation amount TRA of the insertion section 11 and a present rotation amount CRA of the insertion section 11 together with the insertion length meter MIA. In other words, a display example of the operation amount display region SDA shown in FIG. 7 indicates a case in which an insertion length and a rotation amount of the insertion section 11 are used as parameters for operation check and threshold determination by a target insertion length and a target rotation amount of the insertion section 11 is performed.

Note that, according to the present embodiment, for example, at least a part of information included in the respective display regions of the display image DGA may be displayed using a display method corresponding to VR (virtual reality), AR (augmented reality), or MR (mixed reality).

The voice generating unit 260 generates voice corresponding to the guide information included in the operation support information outputted from the system control unit 270 and performs an operation for outputting the generated voice to the outside of the main body apparatus 20 at every predetermined time.

More specifically, for example, the voice generating unit 260 generates voice corresponding to a character string currently displayed in the guide content display region GDD of the display image DGA and performs an operation for outputting the generated voice to the outside of the main body apparatus 20 at every predetermined time.

The examination-support-information generating unit 272 performs, based on the examination situation information acquired by the examination-situation-information acquiring unit 271, for example, every time an endoscopic image for one frame is outputted from the image processing unit 220, processing for determining whether it is necessary to update the operation support information generated in step S3 in FIG. 6 (currently being outputted) (step S4 in FIG. 6).

More specifically, when detecting, based on the examination situation information acquired by the examination-situation-information acquiring unit 271, for example, with threshold determination of a parameter for operation check, that, for example, in a state in which a present examination situation specified from the examination situation information is maintained in one examination situation among respective examination situations defined by the table data TDA shown in FIG. 4, operation corresponding to the guide information included in the operation support information generated in step S3 in FIG. 6 has ended, the examination-support-information generating unit 272 estimates that the operation is completed and acquires a determination result that it is necessary to update the operation support information generated in step S3 in FIG. 6. In a state in which the examination-support-information generating unit 272 detects, based on the examination situation information acquired by the examination-situation-information acquiring unit 271, for example, with the threshold determination of the parameter for operation check, that, for example, the operation corresponding to the guide information included in the operation support information generated in step S3 in FIG. 6 has not ended, when detecting that the present examination situation specified from the examination situation information has changed from one examination situation among the respective examination situations defined by the table data TDA in FIG. 4 to another examination situation, the examination-support-information generating unit 272 estimates that the operation is not completed and acquires a determination result that it is necessary to update the operation support information generated in step S3 in FIG. 6. When detecting, based on the examination situation information acquired by the examination-situation-information acquiring unit 271, for example, with the threshold determination of the parameter for operation check, that, for example, in a state in which the present examination situation specified from the examination situation information is maintained in one examination situation among the respective examination situations defined by the table data TDA shown in FIG. 4, the operation corresponding to the guide information included in the operation support information generated in step S3 in FIG. 6 has not ended, the examination-support-information generating unit 272 estimates that the operation is being performed and acquires a determination result that it is unnecessary to update the operation support information generated in step S3 in FIG. 6.

When obtaining the determination result that it is necessary to update the operation support information generated in step S3 in FIG. 6 (S4: YES), after performing processing for causing the recording unit 273 to record, together with the operation support time information, information indicating an estimation result obtained by estimating whether the operation corresponding to the guide information included in the operation support information is completed, the examination-support-information generating unit 272 performs processing in step S5 in FIG. 6 explained below. When obtaining the determination result that it is unnecessary to update the operation support information generated in step S3 in FIG. 6 (S4: NO), after outputting the operation support information to the display control unit 250 and the voice generating unit 260, the examination-support-information generating unit 272 performs the processing in step S4 in FIG. 6 again.

Note that it is assumed that the examination-support-information generating unit 272 shifts to the processing in step S5 in FIG. 6 explained below not only when obtaining the determination result that it is necessary to update the operation support information generated in step S3 in FIG. 6 but also, for example, when detecting that a predetermined time has elapsed after acquiring the determination result that it is unnecessary to update the operation support information or detecting that the processing in step S4 in FIG. 6 is performed a predetermined number of times.

For example, immediately after completing removal of the insertion section 11 inserted into the large intestine of the subject, by operating an examination end switch (not shown) of the input apparatus 50, the user performs an instruction for stopping the operation relating to the support of the endoscopic examination for the subject.

The examination-situation-information acquiring unit 271 and the examination-support-information generating unit 272 perform termination processing for the loop LA (step S5 in FIG. 6). More specifically, for example, when failing in detecting the instruction from the examination end switch of the input apparatus 50, the examination-situation-information acquiring unit 271 and the examination-support-information generating unit 272 return to step S1 in FIG. 6 and perform the processing of the loop LA again. For example, when detecting the instruction from the examination end switch of the input apparatus 50, the examination-situation-information acquiring unit 271 and the examination-support-information generating unit 272 end the processing of the loop LA and end a series of processing shown in FIG. 6.

When detecting the instruction from the examination end switch of the input apparatus 50, the recording unit 273 stops the operation for recording the endoscopic image outputted from the image processing unit 220, the insertion position information outputted from the insertion shape observing apparatus 40, the insertion shape image outputted from the insertion-shape-image generating unit 240, and the output time and the output content of the operation support information outputted from the examination-support-information generating unit 272 to the display control unit 250.

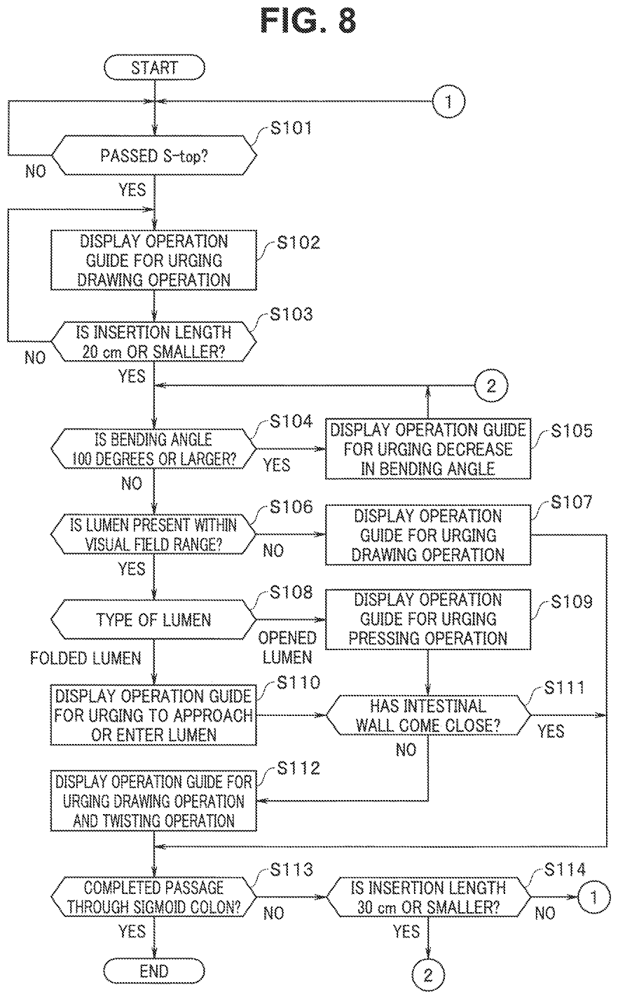

Subsequently, a specific example of the processing in step S3 and step S4 in FIG. 6 repeatedly performed during the endoscopic examination is explained with reference to a flowchart of FIG. 8 and the like. Note that, in the following explanation, an example is explained in which operation conforming to a shaft retention and shortening method is performed as the inserting operation for the insertion section 11 at a passage time of a sigmoid colon. In the following explanation, it is assumed that a lumen of a type equivalent to a closed lumen is absent. FIG. 8 is a flowchart for explaining an example of processing performed in the endoscope system according to the first embodiment.

The examination-support-information generating unit 272 performs, based on examination situation information acquired by the examination-situation-information acquiring unit 271, processing for determining whether the distal end portion 12 has passed an S-top equivalent to a top of the sigmoid colon (step S101 in FIG. 8).

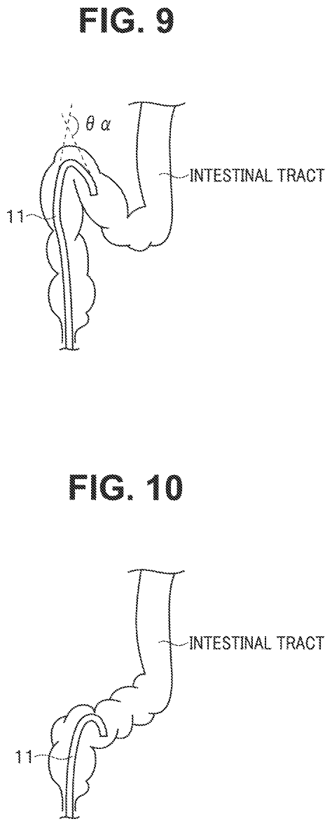

More specifically, when detecting, based on the examination situation information acquired by the examination-situation-information acquiring unit 271, that, for example, a present bending angle of the bending section 13 equivalent to an angle θα shown in FIG. 9 is 100 degrees or larger and that a lumen is present within the visual field range of the image pickup unit 110, the examination-support-information generating unit 272 obtains a determination result that the distal end portion 12 has not passed the S-top. When detecting, based on the examination situation information acquired by the examination-situation-information acquiring unit 271, that, for example, the present bending angle of the bending section 13 is smaller than 100 degrees and that a lumen is absent within the visual field range of the image pickup unit 110, the examination-support-information generating unit 272 obtains a determination result that the distal end portion 12 has not passed the S-top. FIG. 9 is a diagram schematically showing an example of a state of an intestinal tract in a case in which operation of the insertion section is performed according to the processing shown in FIG. 8.

When obtaining the determination result that the distal end portion 12 has not passed the S-top (S101: NO), for example, after generating operation support information including guide information for displaying an operation guide for urging pressing operation equivalent to operation for moving the insertion section 11 forward, the examination-support-information generating unit 272 performs the processing in step S101 in FIG. 8 again. When obtaining the determination result that the distal end portion 12 has passed the S-top (S101: YES), after generating and outputting operation support information including guide information for displaying an operation guide for urging drawing operation equivalent to operation for moving the insertion section 11 backward (step S102 in FIG. 8), the examination-support-information generating unit 272 subsequently performs processing in step S103 in FIG. 8 explained below.

Note that, according to the present embodiment, for example, when, according to a detection result that the present bending angle of the bending section 13 is smaller than 100 degrees and a lumen is present within the visual field range of the image pickup unit 110, obtaining, in step S101 in FIG. 8, the determination result that the distal end portion 12 has not passed the S-top, the examination-support-information generating unit 272 may generate operation support information for not displaying the operation guide relating to the operation of the insertion section 11. According to the present embodiment, for example, when specifying in step S101 in FIG. 8 that the present bending angle of the bending section 13 is 100 degrees or larger and that a lumen is absent within the visual field range of the image pickup unit 110, the examination-support-information generating unit 272 may generate operation support information including guide information for displaying an operation guide for urging operation for disposing the distal end portion 12 in a position where a lumen can be visually recognized.

The examination-support-information generating unit 272 performs, based on the examination situation information acquired by the examination-situation-information acquiring unit 271, processing for determining whether a present insertion length of the insertion section 11 is 20 cm or smaller (step S103 in FIG. 8).

When obtaining a determination result that the present insertion length of the insertion section 11 is 20 cm or smaller (S103: YES), the examination-support-information generating unit 272 subsequently performs processing in step S104 in FIG. 8 explained below. When obtaining a determination result that the present insertion length of the insertion section 11 is larger than 20 cm (S103: NO), after outputting again the operation support information generated in step S102 in FIG. 8 (including the guide information for displaying the operation guide for urging the drawing operation), the examination-support-information generating unit 272 performs the processing in step S103 in FIG. 8 again.

When operation corresponding to the processing in step S101 to step S103 in FIG. 8 is performed by the user, for example, an intestinal tract having a shape shown in FIG. 9 is drawn to a near side (an anus side) and is deformed into a shape shown in FIG. 10. FIG. 10 is a diagram schematically showing an example of a state of the intestinal tract in a case in which operation of the insertion section is performed according to the processing shown in FIG. 8.

The examination-support-information generating unit 272 performs, based on the examination situation information acquired by the examination-situation-information acquiring unit 271, processing for determining whether a present bending angle of the bending section 13 is 100 degrees or larger (step S104 in FIG. 8).

When obtaining a determination result that the present bending angle of the bending section 13 is smaller than 100 degrees (S104: NO), the examination-support-information generating unit 272 subsequently performs processing in step S106 in FIG. 8 explained below. When obtaining a determination result that the present bending angle of the bending section 13 is 100 degrees or larger (S104: YES), after generating operation support information including guide information for displaying an operation guide for urging operation for reducing a bending angle of the bending section 13 (step S106 in FIG. 8), the examination-support-information generating unit 272 performs the processing in step S104 in FIG. 8 again.

The examination-support-information generating unit 272 performs, based on the examination situation information acquired by the examination-situation-information acquiring unit 271, processing for determining whether a lumen is present within the visual field range of the image pickup unit 110 (step S106 in FIG. 8).

When obtaining a determination result that a lumen is present within the visual field range of the image pickup unit 110 (S106: YES), the examination-support-information generating unit 272 subsequently performs processing in step S108 in FIG. 8 explained below. When obtaining a determination result that a lumen is absent within the visual field range of the image pickup unit 110 (S106: NO), after generating operation support information including guide information for displaying an operation guide for urging drawing operation (step S107 in FIG. 8), the examination-support-information generating unit 272 subsequently performs processing in step S113 in FIG. 8 explained below.

The examination-support-information generating unit 272 performs, based on the examination situation information acquired by the examination-situation-information acquiring unit 271, processing for determining a type of the lumen present within the visual field range of the image pickup unit 110 (step S108 in FIG. 8).

When obtaining a determination result that a type of the lumen present within the visual field range of the image pickup unit 110 is an opened lumen, after generating operation support information including guide information for displaying an operation guide for urging pressing operation (step S109 in FIG. 8), the examination-support-information generating unit 272 subsequently performs processing in step S111 in FIG. 8 explained below. When obtaining a determination result that the type of the lumen present within the visual field range of the image pickup unit 110 is a folded lumen, after generating operation support information including guide information for displaying an operation guide for urging operation for causing the distal end portion 12 to approach or enter the lumen (step S110 in FIG. 8), the examination-support-information generating unit 272 subsequently performs the processing in S111 in FIG. 8 explained below.

The examination-support-information generating unit 272 performs, based on the examination situation information acquired by the examination-situation-information acquiring unit 271, processing for determining whether an intestinal wall within the visual field range of the image pickup unit 110 has come close (to the distal end portion 12 side) (step S111 in FIG. 8).

When obtaining a determination result that the intestinal wall within the visual field range of the image pickup unit 110 has come close (to the distal end portion 12 side) (S111: YES), the examination-support-information generating unit 272 subsequently performs processing in step S113 in FIG. 8 explained below. When obtaining a determination result that the intestinal wall within the visual field range of the image pickup unit 110 has not come close (to the distal end portion 12 side) (S111: NO), after generating operation support information including guide information for displaying an operation guide for urging the drawing operation and twisting operation equivalent to operation for rotating the insertion section 11 around the insertion axis (the longitudinal axis) (step S112 in FIG. 8), the examination-support-information generating unit 272 subsequently performs the processing in step S113 in FIG. 8 explained below.

The examination-support-information generating unit 272 performs, based on the examination situation information acquired by the examination-situation-information acquiring unit 271, processing for determining whether the distal end portion 12 has completed passage through the sigmoid colon (step S113 in FIG. 8).

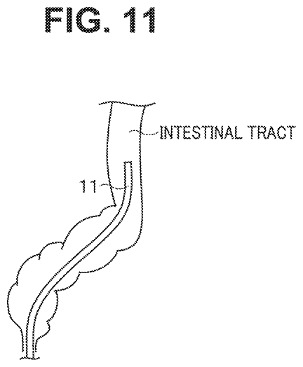

More specifically, when detecting, based on the examination situation information acquired by the examination-situation-information acquiring unit 271, that, for example, an anatomical site corresponding to the visual field range of the image pickup unit 110 is a descending colon, a present insertion length of the insertion section 11 is 25 cm or larger, and the insertion section 11 is straightened (see FIG. 11), the examination-support-information generating unit 272 obtains a determination result that the distal end portion 12 has completed the passage through the sigmoid colon. When detecting, based on the examination situation information acquired by the examination-situation-information acquiring unit 271, at least one of facts that, for example, the anatomical site corresponding to the visual field range of the image pickup unit 110 is not the descending colon, the present insertion length of the insertion section 11 is smaller than 25 cm, and the insertion section 11 is not straightened, the examination-support-information generating unit 272 obtains a determination result that the distal end portion 12 has not completed the passage through the sigmoid colon. FIG. 11 is a diagram schematically showing an example of a state of the intestinal tract in a case in which operation of the insertion section is performed according to the processing shown in FIG. 8.

When obtaining the determination result that the distal end portion 12 has not completed the passage through the sigmoid colon (S113: NO), the examination-support-information generating unit 272 subsequently performs processing in step S114 in FIG. 8 explained below. When obtaining the determination result that the distal end portion 12 has completed the passage through the sigmoid colon (S113: YES), after ending a series of processing shown in FIG. 8, the examination-support-information generating unit 272 shifts to processing relating to generation of operation support information corresponding to the descending colon and a site following the descending colon.

The examination-support-information generating unit 272 performs, based on the examination situation information acquired by the examination-situation-information acquiring unit 271, processing for determining whether the present insertion length of the insertion section 11 is 30 cm or smaller (step S114 in FIG. 8).

When obtaining a determination result that the present insertion length of the insertion section 11 is larger than 30 cm (S114: NO), the examination-support-information generating unit 272 returns to step S101 in FIG. 8 and performs the processing. When obtaining a determination result that the present insertion length of the insertion section 11 is 30 cm or smaller (S114: YES), the examination-support-information generating unit 272 returns to step S104 in FIG. 8 and performs the processing.

Note that, in the present embodiment, the examination-support-information generating unit 272 performs, in a period in which the processing of the loop LA is performed, processing for determining whether the insertion section 11 is buckled, processing for determining whether the insertion section 11 forms a stick shape, and processing for determining whether an estimated pressing force applied to the intestinal wall by the insertion section 11 is excessive. Accordingly, in the present embodiment, when obtaining, during the endoscopic examination, a determination result that the insertion section 11 is buckled, the examination-support-information generating unit 272 performs processing for generating and outputting operation support information including guide information for displaying an operation guide for urging operation for eliminating the buckling. In the present embodiment, when obtaining, during the endoscopic examination, a determination result that the insertion section 11 forms a stick shape, the examination-support-information generating unit 272 performs processing for generating and outputting operation support information including guide information for displaying an operation guide for urging operation for releasing the stick shape. In the present embodiment, when obtaining, during the endoscopic examination, a determination result that the estimated pressing force applied to the intestinal wall by the insertion section 11 is excessive, the examination-support-information generating unit 272 performs processing for generating and outputting operation support information including guide information for displaying an operation guide for urging operation for reducing the estimated pressing force.

For example, at any timing after operating the examination end switch of the input apparatus 50, the user operates a procedure evaluation display switch (not shown) of the input apparatus 50 to thereby perform an instruction for displaying an evaluation result of a procedure performed during the endoscopic examination.

When detecting the instruction from the procedure evaluation display switch of the input apparatus 50, the procedure evaluating unit 274 generates procedure evaluation information based on the endoscopic examination information recorded by the recording unit 273 and outputs the generated procedure evaluation information to the display control unit 250.

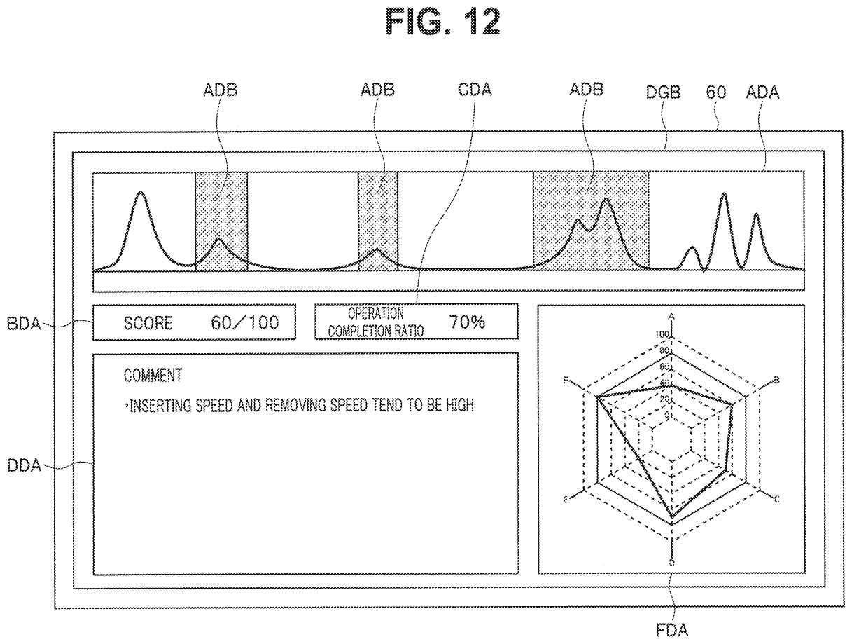

The display control unit 250, based on the procedure evaluation information outputted from the procedure evaluating unit 274, for example, performs processing for generating a display image DGB shown in FIG. 12 and performs processing for causing the display apparatus 60 to display the generated display image DGB. FIG. 12 is a diagram showing an example of a display image displayed by processing of the endoscope system according to the first embodiment.

The display image DGB includes a time-series information display region ADA, a procedure evaluation value display region BDA, an operation completion ratio display region CDA, a comment display region DDA, and an operation level display region FDA.

The time-series information display region ADA is set as a region for displaying a graph showing a temporal change of one evaluation indicator among respective evaluation indicators included in procedure evaluation information outputted from the procedure evaluating unit 274 after an end of the endoscopic examination. For example, as shown in FIG. 12, time-period-of-attention information ADB, which is information indicating a time period when it is estimated that dangerous operation or special operation among respective kinds of operation corresponding to guide information included in operation support information generated by the examination-support-information generating unit 272 is performed during the endoscopic examination, is added to the graph in the time-series information display region ADA.

More specifically, examples of the time period when the time-period-of-attention information ADB is added include a time period when operation conforming to the shaft retention and shortening method was performed on the insertion section 11 inserted into an intestinal tract, a time period when operation for straightening a traverse colon in the intestinal tract was performed, a time period when operation relating to passage through a splenic flexure in the intestinal tract was performed, a time period when operation relating to passage through a hepatic flexure in the intestinal tract was performed, a time period when operation for eliminating bending of the insertion section 11 inserted into the intestinal tract was performed, a time period when operation for releasing a stick shape formed by the insertion section 11 inserted into the intestinal tract was performed, a time period when it was detected that an estimated pressing force applied to an intestinal wall by the insertion section 11 inserted into the intestinal tract was excessive, and a time period when a lumen is absent within the visual field range of the image pickup unit 110 provided in the insertion section 11 inserted into the intestinal tract.

Note that, in the present embodiment, for example, the procedure evaluating unit 274 may perform processing for displaying a graph showing a state in which one evaluation indicator among respective evaluation indicators included in the procedure evaluation information outputted from the procedure evaluating unit 274 changes according to an insertion length of the insertion section 11. In the present embodiment, for example, when operation for selecting one time-period-of-attention information ADB in the time-series information display region ADA is performed, the procedure evaluating unit 274 may perform processing for causing the display apparatus 60 to display, as movies, an endoscopic image and an insertion shape image recorded by the recording unit 273 in a period corresponding to the one time-period-of-attention information ADB.

The procedure evaluation value display region BDA is set as, for example, a region for displaying a procedure evaluation value such as a procedure score indicating a comprehensive evaluation of a procedure performed during the endoscopic examination.

An example of a calculation method for a procedure score displayed in the procedure evaluation value display region BDA is explained. Note that, in the following explanation, for simplification, an example is explained in which an analysis value SPV corresponding to an evaluation indicator SP is included in an analysis result obtained by the endoscopic-image-analysis processing unit 311 and an analysis value SQV corresponding to an evaluation indicator SQ is included in an analysis result obtained by the insertion-shape-analysis processing unit 312.

The evaluation processing unit 313 performs processing for respectively setting a maximum score SMAX equivalent to a maximum value of a procedure score SSV indicating a comprehensive evaluation of a procedure performed during the endoscopic examination, an allocated point AMP of the evaluation indicator SP in the maximum score SMAX, and an allocated point AMQ of the evaluation indicator SQ in the maximum score SMAX.

Note that the allocated point AMP and the allocated point AMQ may be set as, for example, values corresponding to levels of relative importance between the evaluation indicator SP and the evaluation indicator SQ or may be set as values obtained by equally dividing the maximum score SMAX into two.

For example, the evaluation processing unit 313 reads, from the storage medium 20M, distribution information JSP including parameters representing a distribution state of analysis values of the evaluation indicator SP acquired in endoscopic examinations in the past (an average value and a standard deviation of the analysis values of the evaluation indicator SP) and performs an arithmetic operation using the distribution information JSP and the analysis value SPV to thereby calculate a deviation value HPV.

For example, the evaluation processing unit 313 reads, from the storage medium 20M, distribution information JSQ including parameters representing a distribution state of analysis values of the evaluation indicator SQ acquired in endoscopic examinations in the past (an average value and a standard deviation of the analysis values of the evaluation indicator SQ) and performs an arithmetic operation using the distribution information JSQ and an analysis value SPQ to thereby calculate a deviation value HPQ.

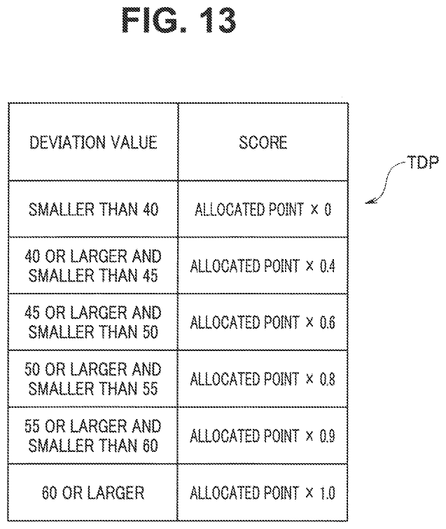

For example, the evaluation processing unit 313 reads, from the storage medium 20M, table data TDP indicating a correspondence relation between a deviation value and a score shown in FIG. 13 and performs an arithmetic operation using the read table data TDP and the allocated points AMP and AMQ to thereby respectively acquire a score PVS corresponding to the deviation value HPV and a score QVS corresponding to the deviation value HPQ.

A “deviation value” field of the table data TDP shown in FIG. 13 includes a plurality of items indicating a range of a deviation value calculated by the evaluation processing unit 313. A “score” field of the table data TDP shown in FIG. 13 includes a plurality of items including information capable of specifying a method of calculating a score using an allocated point set by the evaluation processing unit 313, a coefficient such as a score ratio set in advance for each range of the deviation value shown in the “deviation value” field.

The evaluation processing unit 313 adds up the score PVS and the score QVS acquired as explained above to thereby calculate the procedure score SSV and generates procedure evaluation information including the calculated procedure score SSV and outputs the procedure evaluation information to the display control unit 250. FIG. 13 is a diagram for explaining an example of table data used for processing performed in the endoscope system according to the first embodiment.

Note that, in the present embodiment, not only the procedure score SSV calculated by the method explained above is displayed in the procedure evaluation value display region BDA but also, for example, a procedure evaluation value obtained by evaluating a difference between the procedure score SSV and the maximum score SMAX in a multistage evaluation may be displayed in the procedure evaluation value display region BDA.

Operation completion ratio display region CDA is set as, for example, a region for displaying an operation completion ratio equivalent to a ratio of completing operation corresponding to respective operation guides displayed on the display apparatus 60 during the endoscopic examination.

In other words, the procedure evaluating unit 274 calculates, based on the information indicating the estimation result recorded in the recording unit 273 by the processing of the examination-support-information generating unit 272, an operation completion ratio equivalent to a ratio of completing operation corresponding to each piece of guide information presented during the endoscopic examination and generates procedure evaluation information including information indicating the calculated operation completion ratio and outputs the procedure evaluation information to the display control unit 250.

The comment display region DDA is set as a region for displaying a comment including a character string relating to evaluation of a procedure performed during the endoscopic examination. The comment displayed in the comment display region DDA is set in advance as, for example, a character string corresponding to a type of an evaluation indicator included in the procedure evaluation information outputted from the procedure evaluating unit 274 after the end of the endoscopic examination and an analysis value of the evaluation indicator.

The operation level display region FDA is set as a region for displaying a graph showing a difference between an analysis value and a target value in an evaluation indicator included in the procedure evaluation information outputted from the procedure evaluating unit 274 after the end of the endoscopic examination. More specifically, for example, a radar chart indicating a difference between an analysis value and a target value for each evaluation indicator in a plurality of evaluation indicators included in the procedure evaluation information outputted from the procedure evaluating unit 274 can be displayed in the operation level display region FDA.

Note that, according to the present embodiment, the target value used in displaying the graph in the operation level display region FDA only has to be set as, for example, an average value of analysis values obtained at a time of an endoscopic examination by an expert. According to the present embodiment, for example, a graph showing a difference between a latest analysis value and an analysis value in the past in the evaluation indicator included in the procedure evaluation information outputted from the procedure evaluating unit 274 may be displayed in the operation level display region FDA.

According to the present embodiment, for example, at least a part of information included in respective display regions of the display image DGB may be displayed using a display method corresponding to VR (virtual reality), AR (augmented reality), or MR (mixed reality).

As explained above, according to the present embodiment, it is possible to present an operation method for the insertion section 11 corresponding to a present examination situation in an endoscopic examination to a user. According to the present embodiment, it is possible to present an evaluation for a procedure performed during the endoscopic examination to the user after an end of the endoscopic examination. Accordingly, according to the present embodiment, it is possible to further improve examination quality in the endoscopic examination than in the past.

Note that, according to the present embodiment, for example, when it is possible to perform, with operation of the input apparatus 50, an instruction for alternatively selecting an execution mode equivalent to an endoscopic examination for a human body and a training mode equivalent to an endoscopic examination for a colon model, processing for differentiating display content relating to an evaluation result of a procedure may be performed. More specifically, for example, when the training mode is selected, an operation completion ratio may be displayed in the operation completion ratio display region CDA and, on the other hand, when the execution mode is selected, processing for not displaying the operation completion ratio in the operation completion ratio display region CDA may be performed.

According to the present embodiment, for example, the system control unit 270 may be configured to control, based on the examination situation information acquired by the examination-situation-information acquiring unit 271, an operation of a robot capable of performing operation of the endoscope 10.

According to the present embodiment, for example, the system control unit 270 may, based on the endoscopic examination information recorded by the recording unit 273, be configured as an examination-evaluation-value calculating unit that calculates an examination evaluation value used to evaluate quality of an endoscopic examination and may be configured to perform operation for outputting the calculated examination evaluation value to a database or a Cloud system outside the main body apparatus 20. Further, according to the present embodiment, the system control unit 270 may be configured to calculate, as the examination evaluation value explained above, for example, at least any one of an ileocecum reaching ratio, an intestinal tract cleaning degree, or an adenoma detection rate.

According to the present embodiment, for example, the system control unit 270 may be configured to generate electronic clinical record information based on the endoscopic examination information recorded by the recording unit 273 and perform operation for outputting the generated electronic clinical record information to an electronic clinical record system outside the main body apparatus 20. Further, according to the present embodiment, the system control unit 270 may be configured to generate, as the electronic clinical record information explained above, for example, information including at least one of an endoscopic image obtained by picking up an image of an inside of an intestinal tract, a site where the endoscopic image was obtained, a time point when the endoscopic image was captured, a site where an enlarged observation was used in the intestinal tract, a time point when the enlarged observation was used in the intestinal tract, a site where a narrowband optical observation was used in the intestinal tract, a time point when the narrowband optical observation was used in the intestinal tract, a site where a tissue was sampled in the intestinal tract, a time point when the tissue was sampled in the intestinal tract, a site where a pigment is sprayed in the intestinal tract, a time point when the pigment was sprayed in the intestinal tract, a site where a lesion was detected in the intestinal tract, a time point when the lesion was detected in the intestinal tract, a type of a treatment instrument used in the intestinal tract, or a time point when the insertion section 11 is removed from the intestinal tract.

According to the present embodiment, for example, by combining, based on an endoscopic image outputted from the image processing unit 220, processing for sequentially generating three-dimensional shape data corresponding to a shape of an intestinal tract into which the insertion section 11 is inserted and processing for detecting a motion of the distal end portion 12 corresponding to a temporal change of the endoscopic image, it is possible to realize at least a part of respective functions realized by the examination-situation-information acquiring unit 271 and the procedure evaluating unit 274. Note that, in the present embodiment, as the processing for generating the three-dimensional shape data explained above, for example, processing conforming to a method of Structure from Motion only has to be used. In the present embodiment, as the processing for detecting the motion of the distal end portion 12 explained above, for example, processing conforming to a method of optical flow only has to be used.

According to the present embodiment, for example, respective endoscopic images recorded by the recording unit 273 during the endoscopic examination may be used as images for learning at a time of creation of a classifier having the same function as the function of the recognizer CLP.

According to the present embodiment, for example, the respective endoscopic images recorded by the recording unit 273 during the endoscopic examination may be used as images for learning at a time of creation of a classifier having the same function as the function of the recognizer CLQ.

According to the present embodiment, for example, a plurality of table data having the same configuration as the configuration of the table data TDA shown in FIG. 4 and set to present guide information in a plurality of patterns different for each doctor or each organization may be stored in the storage medium 20M. In such a case, for example, the examination-support-information generating unit 272 may, based on an instruction from the input apparatus 50 or examination situation information acquired by the examination-situation-information acquiring unit 271, perform processing for selecting one table data among the plurality of table data stored by the storage medium 20M and generating operation support information including guide information acquired from the selected one table data.

According to the present embodiment, for example, a plurality of table data having the same configuration as the configuration of the table data TDA shown in FIG. 4 and set to present guide information in a plurality of patterns different for each operation difficulty relating to inserting operation for the insertion section 11. In such a case, for example, the examination-support-information generating unit 272 may, based on an instruction from the input apparatus 50 or examination situation information acquired by the examination-situation-information acquiring unit 271, select one table data among the plurality of table data stored by the storage medium 20M and perform processing for generating operation support information including guide information acquired from the selected one table data. In other words, the examination-support-information generating unit 272 may be configured to perform processing for presenting guide information in patterns different for each operation difficulty relating to the inserting operation for the insertion section 11.

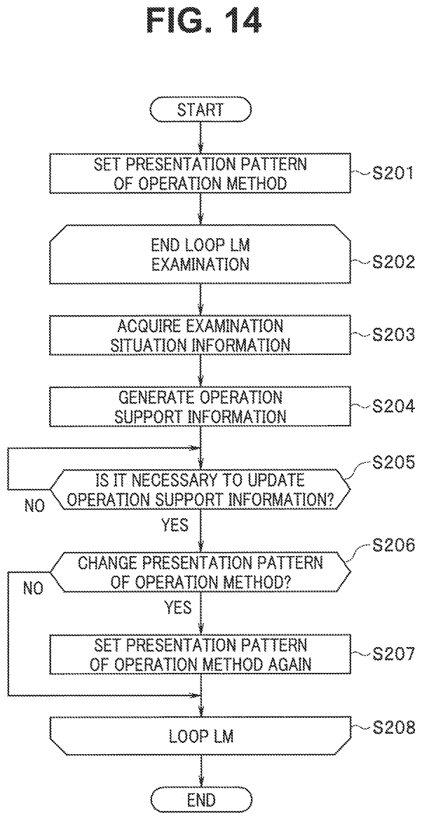

Processing relating to a modification of the present embodiment is explained with reference to FIG. 14. FIG. 14 is a flowchart for explaining an example different from the example shown in FIG. 6 of processing performed in the endoscope system according to the first embodiment.

Note that, in the following explanation, for simplification, specific explanation concerning portions to which the operations and the like explained above are applicable is omitted as appropriate. In the following explanation, an example is explained in which two table data, that is, table data TDN for normal difficulty set to present guide information in a pattern corresponding to normal-difficulty inserting operation for the insertion section 11 and table data TDV for high difficulty set to present guide information in a pattern corresponding to high-difficulty inserting operation for the insertion section 11, are stored in the storage medium 20M and one table data of the two table data is selected based on examination situation information acquired by the examination-situation-information acquiring unit 271.

After connecting the respective sections of the endoscope system 1 and turning on the endoscope system 1, the user operates the input apparatus 50 to thereby perform, for example, an instruction for presenting an operation method in a pattern corresponding to high-difficulty inserting operation for the insertion section 11.

According to the instruction from the input apparatus 50, the examination-support-information generating unit 272 reads the table data TDV for high difficulty from the storage medium 20M to thereby set a presentation pattern for an operation method relating to inserting operation for the insertion section 11 to a high-difficulty pattern (step S201 in FIG. 14).

After performing the instruction relating to the presentation pattern of the operation method of the insertion section 11, the user starts operation for inserting the insertion section 11 from an anus into an inside of a large intestine of a subject. For example, immediately after starting the insertion of the insertion section 11 into the large intestine of the subject, the user operates the examination start switch of the input apparatus 50 to thereby perform an instruction for starting an operation relating to support of an endoscopic examination for the subject.

When detecting the instruction from the examination start switch of the input apparatus 50, the examination-situation-information acquiring unit 271 and the examination-support-information generating unit 272 start processing of a loop LM, which is processing for performing operation support corresponding to a present examination situation in the endoscopic examination (step S202 in FIG. 14).

The examination-situation-information acquiring unit 271 performs, based on an endoscopic image outputted from the image processing unit 220, an insertion shape image outputted from the insertion-shape-image generating unit 240, and insertion position information outputted from the insertion shape observing apparatus 40, processing for acquiring examination situation information indicating a present examination situation in the endoscopic examination (step S203 in FIG. 14).

The examination-support-information generating unit 272 performs processing for generating operation support information based on the examination situation information acquired by the examination-situation-information acquiring unit 271 and performs processing for outputting the generated operation support information to the display control unit 250 and the voice generating unit 260 (step S204 in FIG. 14).

The display control unit 250, based on the endoscopic image outputted from the image processing unit 220 and operation support information outputted from the system control unit 270, performs processing for generating a display image DGM that can include information different from the information included in the display image DGA shown in FIG. 7 and performs processing for causing the display apparatus 60 to display the generated display image DGM.

The display image DGM is generated as, for example, in the guide type display region GDC, an image in which a character string indicating a type of an operation guide further subdivided than the type of the operation guide displayed in the display image DGA shown in FIG. 7 is displayed and an operation method stricter than the operation method displayed in the display image DGA shown in FIG. 7 is displayed.

The voice generating unit 260 generates voice corresponding to guide information included in the operation support information outputted from the system control unit 270 and performs an operation for outputting the generated voice to the outside of the main body apparatus 20 at every predetermined time.

The examination-support-information generating unit 272 performs, based on the examination situation information acquired by the examination-situation-information acquiring unit 271, for example, every time an endoscopic image for one frame is outputted from the image processing unit 220, processing for determining whether it is necessary to update the operation support information generated in step S204 in FIG. 14 (currently being outputted) (step S205 in FIG. 14).

When obtaining a determination result that it is necessary to update the operation support information generated in step S204 in FIG. 14 (S205: YES), after performing processing for causing the recording unit 273 to record, together with operation support time information, information indicating an estimation result obtained by estimating whether operation corresponding to guide information included in the operation support information is completed, the examination-support-information generating unit 272 performs processing in step S206 in FIG. 14 explained below. When obtaining a determination result that it is unnecessary to update the operation support information generated in step S204 in FIG. 14 (S205: NO), after outputting the operation support information to the display control unit 250 and the voice generating unit 260 again, the examination-support-information generating unit 272 performs the processing in step S205 in FIG. 14 again.

Note that it is assumed that the examination-support-information generating unit 272 shifts to the processing in step S206 in FIG. 14 explained below not only when obtaining the determination result that it is necessary to update the operation support information generated in step S204 in FIG. 14 but also when detecting, for example, a predetermined time has elapsed after acquiring the determination result that it is unnecessary to update the operation support information or the processing in step S205 in FIG. 14 is performed a predetermined number of times.

The examination-support-information generating unit 272 performs, based on the information indicating the estimation result recorded by the recording unit 273 according to the processing in step S205 in FIG. 14, processing for determining whether to change a presentation pattern of an operation method currently being set (step S206 in FIG. 14).

More specifically, when detecting, based on the information indicating the estimation result recorded by the recording unit 273 according to the processing in step S205 in FIG. 14, that, for example, the number of times operation corresponding to the guide information included in the table data TDV for high difficulty is not completed is equal to or more than a predetermined number of time, the examination-support-information generating unit 272 acquires a determination result that the presentation pattern of the operation method currently being set is changed. More specifically, when detecting, based on the information indicating the estimation result recorded by the recording unit 273 according to the processing in step S205 in FIG. 14, that, for example, the number of times the operation corresponding to the guide information included in the table data TDV for high difficulty is not completed is less than the predetermined number of time, the examination-support-information generating unit 272 acquires a determination result that the presentation pattern of the operation method currently being set is not changed.

When obtaining the determination result that the presentation pattern of the operation method currently being set is changed (S206: YES), for example, after reading the table data TDN for normal difficulty from the storage medium 20M to thereby set the presentation pattern of the operation method relating to the inserting operation for the insertion section 11 to the normal-difficulty pattern again (step S207 in FIG. 14), the examination-support-information generating unit 272 performs processing in step S208 in FIG. 14 explained below. When obtaining the determination result that the presentation pattern of the operation method currently being set is not changed (S206: NO), the examination-support-information generating unit 272 performs the processing in step S208 in FIG. 14 explained below.

The examination-situation-information acquiring unit 271 and the examination-support-information generating unit 272 perform termination processing for the loop LM (step S208 in FIG. 14). More specifically, for example, when failing in detecting an instruction from the examination end switch of the input apparatus 50, the examination-situation-information acquiring unit 271 and the examination-support-information generating unit 272 return to step S202 in FIG. 14 and perform the processing of the loop LM again. For example, when detecting the instruction from the examination end switch of the input apparatus 50, the examination-situation-information acquiring unit 271 and the examination-support-information generating unit 272 end the processing of the loop LM and end a series of processing shown in FIG. 14.

Note that, in this modification, when the table data TDN for normal difficulty is read by the examination-support-information generating unit 272, for example, processing for generating a display image including the same information as the information included in the display image DGA shown in FIG. 7 is performed by the display control unit 250. In this modification, processing for generating a display image including the same information as the information included in the display image DGB shown in FIG. 12 is performed by the display control unit 250.

As explained above, according to this modification, it is possible to present, to the user, an operation method for the insertion section 11 corresponding to a present examination situation in the endoscopic examination. According to this modification, it is possible to present, to the user, after an end of the endoscopic examination, an evaluation for a procedure performed during the endoscopic examination. Accordingly, according to this modification, it is possible to further improve examination quality in the endoscopic examination than in the past.

### Second Embodiment

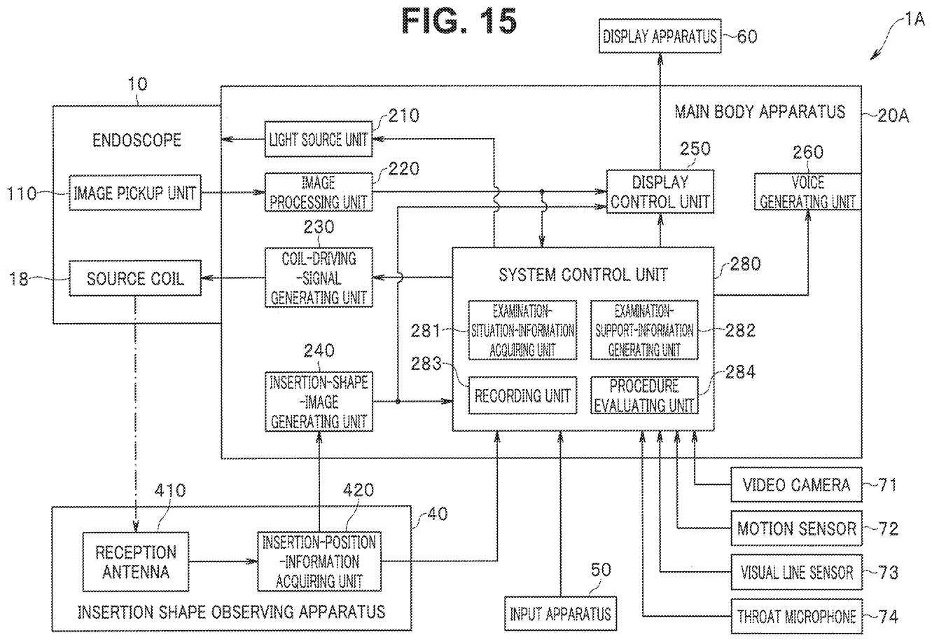

FIG. 15 to FIG. 18 relate to a second embodiment.

Note that, in the present embodiment, detailed explanation concerning portions having the same configurations and the like as the configurations and the like in the first embodiment is omitted as appropriate and portions having configurations and the like different from the configurations and the like in the first embodiment are mainly explained.

An endoscope system 1A includes, for example, as shown in FIG. 15, the endoscope 10, a main body apparatus 20A, the insertion shape observing apparatus 40, the input apparatus 50, the display apparatus 60, a video camera 71, a motion sensor 72, a visual line sensor 73, and a throat microphone 74. FIG. 15 is a block diagram for explaining an example of a configuration of an endoscope system according to the second embodiment.

The main body apparatus 20A includes a system control unit 280 instead of the system control unit 270 in the main body apparatus 20. The main body apparatus 20A includes the one or more processors 20P and the storage medium 20M.

The video camera 71 is disposed in, for example, a position where at least an upper part of a body of a user who operates the endoscope 10 can be photographed. The video camera 71 is configured to photograph the user who is operating the endoscope 10 and acquire a video and output the acquired video to the main body apparatus 20A. Note that, in the present embodiment, a TOF camera or the like may be used instead of the video camera 71.

The motion sensor 72 is configured as, for example, an instrument attachable to both hands or the like of the user who operates the endoscope 10. The motion sensor 72 is configured to acquire motion information equivalent to information capable of specifying a motion of the user who is operating the endoscope 10 and output the acquired information to the main body apparatus 20A.

Note that, in the present embodiment, as a sensor for acquiring motion information of the user who is operating the endoscope 10, for example, a terrestrial magnetism sensor, a press sensor, and a bend sensor may be used.

The visual line sensor 73 is configured as, for example, an instrument attachable to vicinities of both eyes of the user who operates the endoscope 10. The visual line sensor 73 is configured to acquire visual line information equivalent to information capable of specifying a visual line of the user who is operating the endoscope 10 and output the acquired information to the main body apparatus 20A.

The throat microphone 74 is configured as an instrument attachable to a surface of a throat of the user who operates the endoscope 10. The throat microphone 74 is configured to acquire voice corresponding to vibration of the throat of the user who is operating the endoscope 10 and output the acquired voice to the main body apparatus 20A.

The system control unit 280 includes an examination-situation-information acquiring unit 281, an examination-support-information generating unit 282, a recording unit 283, and a procedure evaluating unit 284.

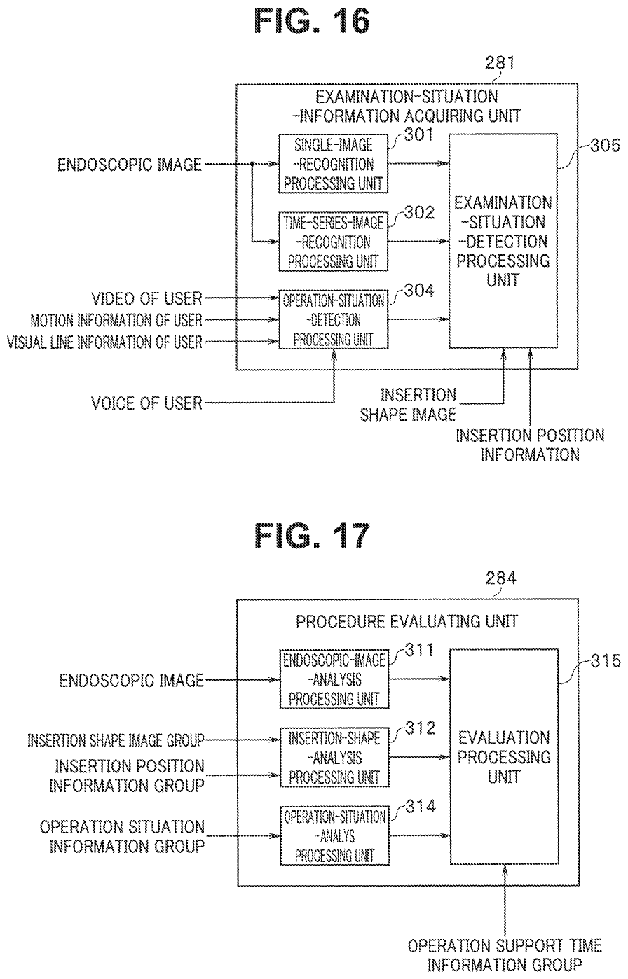

The examination-situation-information acquiring unit 281 is configured to perform, based on an endoscopic image outputted from the image processing unit 220, an insertion shape image outputted from the insertion-shape-image generating unit 240, insertion position information outputted from the insertion shape observing apparatus 40, a video of the user outputted from the video camera 71, motion information of the user outputted from the motion sensor 72, visual line information of the user outputted from the visual line sensor 73, and voice of the user outputted from the throat microphone 74, processing for acquiring examination situation information equivalent to information indicating a present examination situation in the endoscopic examination. More specifically, the examination-situation-information acquiring unit 281 includes, for example, as shown in FIG. 16, a single-image-recognition processing unit 301, a time-series-image-recognition processing unit 302, an operation-situation-detection processing unit 304, and an examination-situation-detection processing unit 305. FIG. 16 is a block diagram for explaining an example of a configuration of an examination-situation-information acquiring unit according to the second embodiment.

The operation-situation-detection processing unit 304 is configured to perform processing based on a detection result obtained by detecting at least one of the video of the user outputted from the video camera 71, the motion information of the user outputted from the motion sensor 72, the visual line information of the user outputted from the visual line sensor 73, or the voice of the user outputted from the throat microphone 74 to thereby acquire operation situation information equivalent to information indicating a present operation situation of the user who is operating the endoscope 10.

More specifically, the operation-situation-detection processing unit 304 is configured to be able to obtain, as the information included in the operation situation information, at least one piece of information among, for example, a positional relation between the endoscope 10 and a body of the user, a positional relation between a left hand and a right hand of the user at a time of operation of the insertion section 11, a way of twisting the insertion section 11 by the user, height of an elbow of the user, height of the right hand of the user based on height of an examination table on which a subject is placed, a motion range of the elbow of the user, a motion range of the hands of the user, presence or absence of operation relating to a change in hardness of the insertion section 11 by the user, or instruction content performed by the user on an supporter who supports the user.

The examination-situation-detection processing unit 305 is configured to perform processing using at least one of insertion position information outputted from the insertion shape observing apparatus 40, a recognition result obtained by the single-image-recognition processing unit 301, a recognition result obtained by the time-series-image-recognition processing unit 302, operation situation information obtained by the operation-situation-detection processing unit 304, or an insertion shape image outputted from the insertion-shape-image generating unit 240 to thereby acquire examination situation information indicating a present examination situation in the endoscopic examination.

The examination-support-information generating unit 282 is configured to perform, based on user information inputted in the input apparatus 50, processing for determining whether a skill level of the user who operates the endoscope 10 is equivalent to an expert skill level.

Note that, in the present embodiment, at least one piece of information among, for example, the number of years of experience of an endoscopic examination in the user who operates the endoscope 10, the number of experienced cases in the user, a diagnosis and treatment department of the user, presence or absence of a certified physician qualification in the user, or presence or absence of a specialist qualification in the user only has to be inputted as the user information explained above. In other words, in the present embodiment, only one or more pieces of information relating to the skill level of the user who operates the endoscope 10 (performs the endoscopic examination) have to be included in the user information explained above. In the present embodiment, for example, the examination-support-information generating unit 282 may perform processing for acquiring the user information from a database or the like outside the main body apparatus 20. According to the present embodiment, for example, when the user who performs the endoscopic examination has a certified physician qualification or a specialist qualification and the number of experienced cases in the user is a predetermined number or more, a determination result that the skill level of the user is equivalent to the expert skill level only has to be obtained. According to the present embodiment, for example, when the user who performs the endoscopic examination has a certified physician qualification or a specialist qualification and, on the other hand, the number of experienced cases in the user is less than the predetermined number, a determination result that the skill level of the user is not equivalent to the expert skill level only has to be obtained.

The examination-support-information generating unit 282 is configured to, when obtaining a determination result that the skill level of the user who operates the endoscope 10 is not equivalent to the expert skill level, based on the examination situation information acquired by the examination-situation-information acquiring unit 281, perform processing for generating operation support information including guide information for nonexperts and perform processing for outputting the generated operation support information to the display control unit 250 and the voice generating unit 260.

More specifically, the examination-support-information generating unit 282 is configured to, when obtaining the determination result that the skill level of the user who operates the endoscope 10 is not equivalent to the expert skill level, read table data TDB for nonexperts from the storage medium 20M, select, out of a plurality of pieces of guide information included in the read table data TDB, one guide information corresponding to a present examination situation specified based on the examination situation information acquired by the examination-situation-information acquiring unit 281, and generate operation support information including the selected one guide information and output the operation support information to the display control unit 250 and the voice generating unit 260.

The table data TDB for nonexperts is created as data obtained by replacing an operation methods in respective items of the “guide information” field of the table data TDA shown in FIG. 4 with a detailed operation method that takes into account an operation situation of the user (for example, “please press while carefully watching a lumen” and “please twist to the right while turning the left wrist”).

Note that, in the present embodiment, for example, when obtaining the determination result that the skill level of the user who operates the endoscope 10 is not equivalent to the expert skill level, the examination-support-information generating unit 282 may further perform processing for determining to which of beginner and intermediate skill levels the skill level of the user is equivalent. In the present embodiment, when obtaining a determination result that the skill level of the user who operates the endoscope 10 is equivalent to the beginner skill level, the examination-support-information generating unit 282 may read table data for beginners from the storage medium 20M and generate operation support information. When obtaining a determination result that the skill level of the user is equivalent to the intermediate skill level, the examination-support-information generating unit 282 may read table data for intermediates from the storage medium 20M and generate operation support information.

The examination-support-information generating unit 282 is configured to, when obtaining the determination result that the skill level of the user who operates the endoscope 10 is equivalent to the expert skill level, based on the examination situation information acquired by the examination-situation-information acquiring unit 281, perform processing for generating operation support information including guide information for experts and perform processing for outputting the generated operation support information to the display control unit 250 and the voice generating unit 260.

More specifically, the examination-support-information generating unit 282 is configured to, when obtaining the determination result that the skill level of the user who operates the endoscope 10 is equivalent to the expert skill level, read table data TDC for experts from the storage medium 20M, select, out of a plurality of pieces of guide information included in the read table data TDC, one guide information corresponding to a present examination situation specified based on the examination situation information acquired by the examination-situation-information acquiring unit 281, and generate operation support information including the selected one guide information and output the operation support information to the display control unit 250 and the voice generating unit 260.

The table data TDC for experts is created as data obtained by deleting the “parameter for operation check” field of the table data TDA and replacing the type of the guide and the operation methods in the respective items of the “guide information” field of the table data TDA shown in FIG. 4 with work content (for example, “executing a first stage of work relating to loop release”) estimated from an operation situation of the user.

In other words, the examination-support-information generating unit 282 is configured to generate, based on the examination situation information acquired by the examination-situation-information acquiring unit 281 and user information including information relating to the skill level of the user who operates the endoscope 10, the operation support information for presenting different guide information according to whether the skill level of the user is equivalent to the expert skill level. The examination-support-information generating unit 282 is configured to, when obtaining, based on the user information inputted in the input apparatus 50, the determination result that the skill level of the user is equivalent to the expert skill level, perform processing for generating operation support information including guide information for presenting work content currently performed by the user.

The examination-support-information generating unit 282 is configured to perform, based on the examination situation information acquired by the examination-situation-information acquiring unit 281, for example, every time an endoscopic image for one frame is outputted from the image processing unit 220, processing for determining whether it is necessary to update operation support information currently being outputted.

More specifically, for example, when acquiring guide information from the table data TDB for nonexperts, the examination-support-information generating unit 282 performs the same processing as the processing performed when the guide information is acquired from the table data TDA to thereby determine whether it is necessary to update the operation support information currently being outputted. For example, when acquiring guide information from the table data TDC for experts, the examination-support-information generating unit 282 determines, based on whether a present examination situation indicated by the examination situation information acquired by the examination-situation-information acquiring unit 281 has changed from one examination situation among respective examination situations defined by the table data TDC to another examination situation, whether it is necessary to update the operation support information currently being outputted.

The examination-support-information generating unit 282 is configured to, when obtaining a determination result that it is necessary to update the operation support information currently being outputted, based on the examination situation information acquired by the examination-situation-information acquiring unit 281, perform processing for generating new operation support information including guide information different from guide information included in the operation support information and perform processing for outputting the generated new operation support information to the display control unit 250 and the voice generating unit 260. The examination-support-information generating unit 282 is configured to, when obtaining a determination result that it is unnecessary to update the operation support information currently being outputted, perform processing for outputting the operation support information to the display control unit 250 and the voice generating unit 260 again.

The recording unit 283 is configured to perform an operation for recording an image, information, and the like obtained in the endoscopic examination as endoscopic examination information.

More specifically, the recording unit 283 performs an operation for respectively recording, during the endoscopic examination, for example, the endoscopic image outputted from the image processing unit 220, the insertion position information outputted from the insertion shape observing apparatus 40, the insertion shape image outputted from the insertion-shape-image generating unit 240, and the operation situation information acquired by the operation-situation-detection processing unit 304. Every time one operation support information is generated by the examination-support-information generating unit 282, the recording unit 283 performs an operation for recording information indicating a time in which the one operation support information is continuously outputted to the display control unit 250.

In other words, the recording unit 283 records, as endoscopic examination information in one endoscopic examination, information correlating an endoscopic image group including endoscopic images for a plurality of frames sequentially outputted from the image processing unit 220 during the one endoscopic examination, an insertion position information group including a plurality of insertion position information sequentially outputted from the insertion shape observing apparatus 40 during the one endoscopic examination, an insertion shape image group including a plurality of insertion shape images sequentially outputted from the insertion-shape-image generating unit 240 during the one endoscopic examination, an operation situation information group including a plurality of pieces of operation situation information acquired according to an action of the user in the one endoscopic examination, and an operation support time information group indicating output times and output contents of a respective plurality of pieces of operation support information outputted from the examination-support-information generating unit 282 to the display control unit 250 during the one endoscopic examination.

The procedure evaluating unit 284 is configured to, based on the endoscopic examination information recorded by the recording unit 283, generate procedure evaluation information including information obtained by evaluating a procedure including inserting operation for the insertion section 11 performed by the user during the endoscopic examination and perform processing for outputting the generated procedure evaluation information to the display control unit 250. More specifically, the procedure evaluating unit 284 includes, for example, as shown in FIG. 17, the endoscopic-image-analysis processing unit 311, the insertion-shape-analysis processing unit 312, an operation-situation-analysis processing unit 314, and an evaluation processing unit 315. FIG. 17 is a block diagram for explaining an example of a configuration of a procedure evaluating unit according to the second embodiment.

The operation-situation-analysis processing unit 314 is configured to perform analysis processing based on the operation situation information group included in the endoscopic examination information recorded by the recording unit 283 to thereby obtain an analysis result including analysis values of one or more evaluation indicators corresponding to an action of the user during the endoscopic examination.

More specifically, by performing the analysis processing based on the operation situation information group included in the endoscopic examination information recorded by the recording unit 283, the operation-situation-analysis processing unit 314 can obtain an analysis result including an analysis value of at least one evaluation indicator among, for example, a distance between the left hand and the right hand of the user who is performing the endoscopic examination, a coordinate value indicating a position of a visual line of the user, or a motion range of the hands of the user.

The evaluation processing unit 315 is configured to perform processing using an analysis result obtained by the endoscopic-image-analysis processing unit 311, an analysis result obtained by the insertion-shape-analysis processing unit 312, an analysis result obtained by the operation-situation-analysis processing unit 314, and the operation support time information group included in the endoscopic examination information recorded by the recording unit 283 to thereby generate procedure evaluation information and perform processing for outputting the generated procedure evaluation information to the display control unit 250.

In the present embodiment, at least a part of functions of the main body apparatus 20A only has to be realized by the processor 20P. In the present embodiment, at least a part of the main body apparatus 20A may be configured as individual electronic circuits or may be configured as a circuit block in an integrated circuit such as an FPGA (field programmable gate array). By modifying the configuration according to the present embodiment as appropriate, for example, a computer may read, from the storage medium 20M such as a memory, a program for executing at least a part of the functions of the main body apparatus 20A and perform an operation corresponding to the read program.

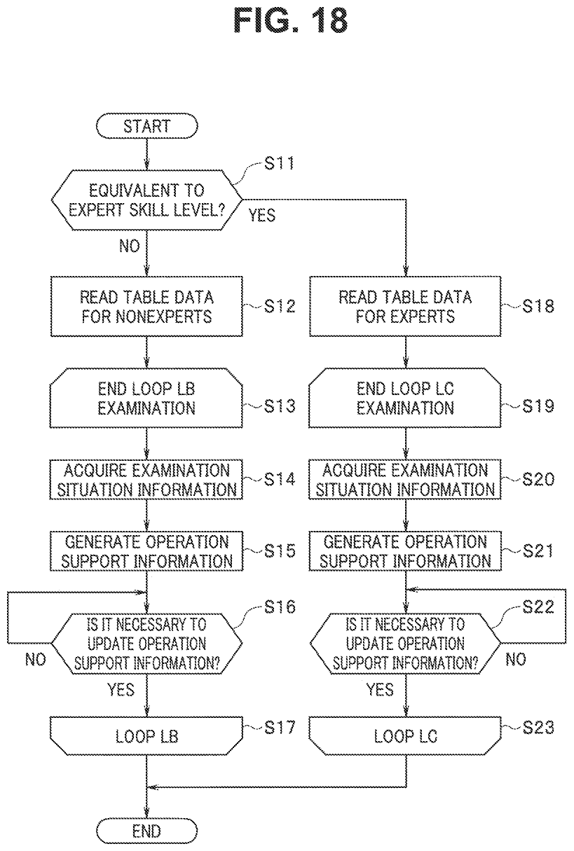

Subsequently, action of the present embodiment is explained with reference to FIG. 18. FIG. 18 is a flowchart for explaining an example of processing performed in the endoscope system according to the second embodiment.

After connecting the respective sections of the endoscope system 1A and turning on the endoscope system 1A, the user operates the input apparatus 50 to thereby input user information used for determination of a skill level of the user.

With the operation of the user explained above, an object is irradiated with illumination light supplied from the light source unit 210, an image of the object irradiated with the illumination light is picked up by the image pickup unit 110, and an endoscopic image obtained by picking up an image of the object is outputted from the image processing unit 220 to the display control unit 250 and the system control unit 280. With the operation of the user explained above, a coil driving signal is supplied from the coil-driving-signal generating unit 230, magnetic fields are emitted from the respective plurality of source coils 18 according to the coil driving signal, insertion position information obtained by detecting the magnetic fields is outputted from the insertion-position-information acquiring unit 420 to the insertion-shape-image generating unit 240 and the system control unit 280, and an insertion shape image generated according to the insertion position information is outputted from the insertion-shape-image generating unit 240 to the system control unit 280. With the operation of the user explained above, a video of the user obtained by the video camera 71, motion information of the user obtained by the motion sensor 72, visual line information of the user obtained by the visual line sensor 73, and voice of the user obtained by the throat microphone 74 are respectively outputted to the examination-situation-information acquiring unit 281.

The examination-support-information generating unit 282 performs, based on the user information inputted in the input apparatus 50, processing for determining whether a skill level of the user who operates the endoscope 10 is equivalent to the expert skill level (step S11 in FIG. 18).

When obtaining a determination result that the skill level of the user who operates the endoscope 10 is not equivalent to the expert skill level (S11: NO), after reading the table data TDB for nonexperts from the storage medium 20M (step S12 in FIG. 18) and performing processing for causing the recording unit 283 to record information indicating the determination result together with operation situation information, the examination-support-information generating unit 282 shifts to processing in step S13 in FIG. 18 explained below. When obtaining a determination result that the skill level of the user who operates the endoscope 10 is equivalent to the expert skill level (S11: YES), after reading the table data TDC for experts from the storage medium 20M (step S18 in FIG. 18) and performing processing for causing the recording unit 283 to record information indicating the determination result together with the operation situation information, the examination-support-information generating unit 282 shifts to processing in step S19 in FIG. 18 explained below.

After inputting the user information, the user starts operation for inserting the insertion section 11 from an anus to an inside of a large intestine of a subject. For example, immediately after starting the insertion of the insertion section 11 into the large intestine of the subject, the user operates the examination start switch of the input apparatus 50 to thereby perform an instruction for starting an operation relating to support of an endoscopic examination for the subject.

When detecting the instruction from the examination start switch of the input apparatus 50 after reading the table data TDB for nonexperts, the examination-situation-information acquiring unit 281 and the examination-support-information generating unit 282 start processing of a loop LB, which is processing for performing operation support corresponding to a present examination situation in the endoscopic examination (step S13 in FIG. 18).

The examination-situation-information acquiring unit 281 performs, based on the endoscopic image outputted from the image processing unit 220, the insertion shape image outputted from the insertion-shape-image generating unit 240, the insertion position information outputted from the insertion shape observing apparatus 40, the video of the user outputted from the video camera 71, the motion information of the user outputted from the motion sensor 72, the visual line information of the user outputted from the visual line sensor 73, and the voice of the user outputted from the throat microphone 74, processing for acquiring examination situation information equivalent to information indicating a present examination situation in the endoscopic examination (step S14 in FIG. 18).

The examination-support-information generating unit 282 performs processing for generating operation support information based on the examination situation information acquired by the examination-situation-information acquiring unit 281 and the table data TDB read in step S12 in FIG. 18 and performs processing for outputting the generated operation support information to the display control unit 250 and the voice generating unit 260 (step S15 in FIG. 18).

The display control unit 250, based on the endoscopic image outputted from the image processing unit 220 and the operation support information outputted from the system control unit 280, for example performs processing for generating a display image including the same information as the information included in the display image DGA shown in FIG. 7 and performs processing for causing the display apparatus 60 to display the generated display image.

The voice generating unit 260 generates voice corresponding to guide information included in the operation support information outputted from the system control unit 280 and performs an operation for outputting the generated voice to the outside of the main body apparatus 20 at every predetermined time.

The examination-support-information generating unit 282 performs, based on the examination situation information acquired by the examination-situation-information acquiring unit 281, for example, every time an endoscopic image for one frame is outputted from the image processing unit 220, processing for determining whether it is necessary to update the operation support information generated in step S15 in FIG. 18 (currently being outputted) (step S16 in FIG. 18).

Note that, in the present embodiment, the same processing as the processing in step S4 in FIG. 6 only has to be performed as the processing in step S16 in FIG. 18.

When obtaining a determination result that it is necessary to update the operation support information generated in step S15 in FIG. 18 (S16: YES), after performing processing for causing the recording unit 283 to record, together with operation support time information, information indicating an estimation result obtained by estimating whether operation corresponding to the guide information included in the operation support information is completed, the examination-support-information generating unit 282 performs processing in step S17 in FIG. 18 explained below. When obtaining a determination result that it is unnecessary to update the operation support information generated in step S15 in FIG. 18 (S16: NO), after outputting the operation support information to the display control unit 250 and the voice generating unit 260 again, the examination-support-information generating unit 282 performs the processing in step S16 in FIG. 18 again.

Note that it is assumed that the examination-support-information generating unit 282 shifts to the processing in step S17 in FIG. 18 explained below not only when obtaining the determination result that it is necessary to update the operation support information generated in step S15 in FIG. 18 but also, for example, when detecting that a predetermined time has elapsed after acquiring the determination result that it is unnecessary to update the operation support information or detecting that the processing in step S16 in FIG. 18 is performed a predetermined number of times.

For example, immediately after completing removal of the insertion section 11 inserted into the large intestine of the subject, by operating the examination end switch of the input apparatus 50, the user performs an instruction for stopping the operation relating to the support of the endoscopic examination for the subject.

The examination-situation-information acquiring unit 281 and the examination-support-information generating unit 282 perform termination processing for the loop LB (step S17 in FIG. 18). More specifically, for example, when failing in detecting the instruction from the examination end switch of the input apparatus 50, the examination-situation-information acquiring unit 281 and the examination-support-information generating unit 282 return to step S13 in FIG. 18 and perform the processing of the loop LB again. For example, when detecting the instruction from the examination end switch of the input apparatus 50, the examination-situation-information acquiring unit 281 and the examination-support-information generating unit 282 end the processing of the loop LB and end a series of processing shown in FIG. 18.

When detecting the instruction from the examination start switch of the input apparatus 50 after reading the table data TDC for experts, the examination-situation-information acquiring unit 281 and the examination-support-information generating unit 282 start processing of a loop LC, which is processing for presenting work content corresponding to a present examination situation in the endoscopic examination (step S19 in FIG. 18).

The examination-situation-information acquiring unit 281 performs, based on the endoscopic image outputted from the image processing unit 220, the insertion shape image outputted from the insertion-shape-image generating unit 240, the insertion position information outputted from the insertion shape observing apparatus 40, the video of the user outputted from the video camera 71, the motion information of the user outputted from the motion sensor 72, the visual line information of the user outputted from the visual line sensor 73, and the voice of the user outputted from the throat microphone 74, processing for acquiring examination situation information equivalent to information indicating a present examination situation in the endoscopic examination (step S20 in FIG. 18).

The examination-support-information generating unit 282 performs processing for generating operation support information based on the examination situation information acquired by the examination-situation-information acquiring unit 281 and the table data TDC read in step S18 in FIG. 18 and performs processing for outputting the generated operation support information to the display control unit 250 and the voice generating unit 260 (step S21 in FIG. 18).

The display control unit 250, based on the endoscopic image outputted from the image processing unit 220 and the operation support information outputted from the system control unit 280, performs processing for generating a display image DGC different from the display image DGA shown in FIG. 7 and performs processing for causing the display apparatus 60 to display the generated display image DGC.

The display image DGC is generated as, for example, an image obtained by deleting the guide type display region GDC, the operation example display region GDE, and the operation amount display region SDA from the display image DGA shown in FIG. 7 and replacing display content of the guide content display region GDD in the display image DGA with work content currently performed by the user.

The examination-support-information generating unit 282 performs, based on the examination situation information acquired by the examination-situation-information acquiring unit 281, for example, every time an endoscopic image for one frame is outputted from the image processing unit 220, processing for determining whether it is necessary to update the operation support information generated in step S21 in FIG. 18 (currently being outputted) (step S22 in FIG. 18).

More specifically, for example, when detecting that the present examination situation indicated by the examination situation information acquired by the examination-situation-information acquiring unit 281 is maintained in one examination situation among the respective examination situations defined by the table data TDC, the examination-support-information generating unit 282 acquires a determination result that it is unnecessary to update the operation support information generated in step S21 in FIG. 18. For example, when detecting that the present examination situation indicated by the examination situation information acquired by the examination-situation-information acquiring unit 281 has changed from one examination situation among the respective examination situations defined by the table data TDC to another examination situation, the examination-support-information generating unit 282 acquires a determination result that it is necessary to update the operation support information generated in step S21 in FIG. 18.

When obtaining the determination result that it is necessary to update the operation support information generated in step S21 in FIG. 18 (S22: YES), the examination-support-information generating unit 282 performs processing in step S23 in FIG. 18 explained below. When obtaining the determination result that it is unnecessary to update the operation support information generated in step S21 in FIG. 18 (S22: NO), after outputting the operation support information to the display control unit 250 and the voice generating unit 260 again, the examination-support-information generating unit 282 performs the processing in step S22 in FIG. 18 again.

Note that it is assumed that the examination-support-information generating unit 282 shifts to the processing in step S23 in FIG. 18 explained below not only when obtaining the determination result that it is necessary to update the operation support information generated in step S21 in FIG. 18 but also, for example, when detecting that a predetermined time has elapsed after acquiring the determination result that it is unnecessary to update the operation support information or detecting that the processing in step S22 in FIG. 18 is performed a predetermined number of times.

For example, immediately after completing removal of the insertion section 11 inserted into the large intestine of the subject, by operating the examination end switch of the input apparatus 50, the user performs an instruction for stopping the operation relating to the support of the endoscopic examination for the subject.

The examination-situation-information acquiring unit 281 and the examination-support-information generating unit 282 perform termination processing for the loop LC (step S23 in FIG. 18). More specifically, for example, when failing in detecting the instruction from the examination end switch of the input apparatus 50, the examination-situation-information acquiring unit 281 and the examination-support-information generating unit 282 return to step S19 in FIG. 18 and perform the processing of the loop LC again. For example, when detecting the instruction from the examination end switch of the input apparatus 50, the examination-situation-information acquiring unit 281 and the examination-support-information generating unit 282 end the processing of the loop LC and end the series of processing shown in FIG. 18.

For example, at any timing after operating the examination end switch of the input apparatus 50, the user operates the procedure evaluation display switch of the input apparatus 50 to thereby perform an instruction for displaying an evaluation result of a procedure performed during the endoscopic examination.

When detecting the instruction from the procedure evaluation display switch of the input apparatus 50, the procedure evaluating unit 284 performs processing for determining from which of a nonexpert or an expert the endoscopic examination information recorded by the recording unit 283 is obtained.

When obtaining a determination result that the endoscopic examination information recorded by the recording unit 283 is obtained by the nonexpert, the procedure evaluating unit 284 generates procedure evaluation information for nonexperts and outputs the procedure evaluation information for nonexperts to the display control unit 250.

The display control unit 250, based on the procedure evaluation information for nonexperts outputted from the procedure evaluating unit 284, for example performs processing for generating a display image including the same information as the information included in the display image DGB shown in FIG. 12 and performs processing for causing the display apparatus 60 to display the generated display image.

When obtaining a determination result that the endoscopic examination information recorded by the recording unit 283 is obtained by the expert, the procedure evaluating unit 284 generates procedure evaluation information for experts and outputs the procedure evaluation information for experts to the display control unit 250.

The display control unit 250, based on the procedure evaluation information for experts outputted from the procedure evaluating unit 284, for example performs processing for generating a display image DGD different from the display image DGB shown in FIG. 12 and performs processing for causing the display apparatus 60 to display the generated display image DGD.

The display image DGD is generated as an image obtained by deleting an operation completion ratio display region CDA from the display image DGB shown in FIG. 12.

As explained above, according to the present embodiment, it is possible to present an operation method for the insertion section 11 corresponding to a skill level of a user who performs an endoscopic examination and a present examination situation in the endoscopic examination. According to the present embodiment, it is possible to present an evaluation for a procedure performed during the endoscopic examination to the user after an end of the endoscopic examination. Accordingly, according to the present embodiment, it is possible to further improve examination quality in the endoscopic examination than in the past.

Note that, according to the present embodiment, for example, the examination-support-information generating unit 282 may be configured to, when detecting that information capable of specifying a respective plurality of users is included in the user information inputted in the input apparatus 50, perform processing for causing the recording unit 283 to record endoscopic examination information obtained during the endoscopic examination while distinguishing the endoscopic examination information for each of the users. Further, according to the present embodiment, for example, the system control unit 280 may be configured to perform an operation for outputting the endoscopic examination information, which is recorded by the recording unit 283 in a state in which the endoscopic examination information is distinguished for each of the users, to a database or a Cloud system on the outside of the main body apparatus 20A.

### Third Embodiment

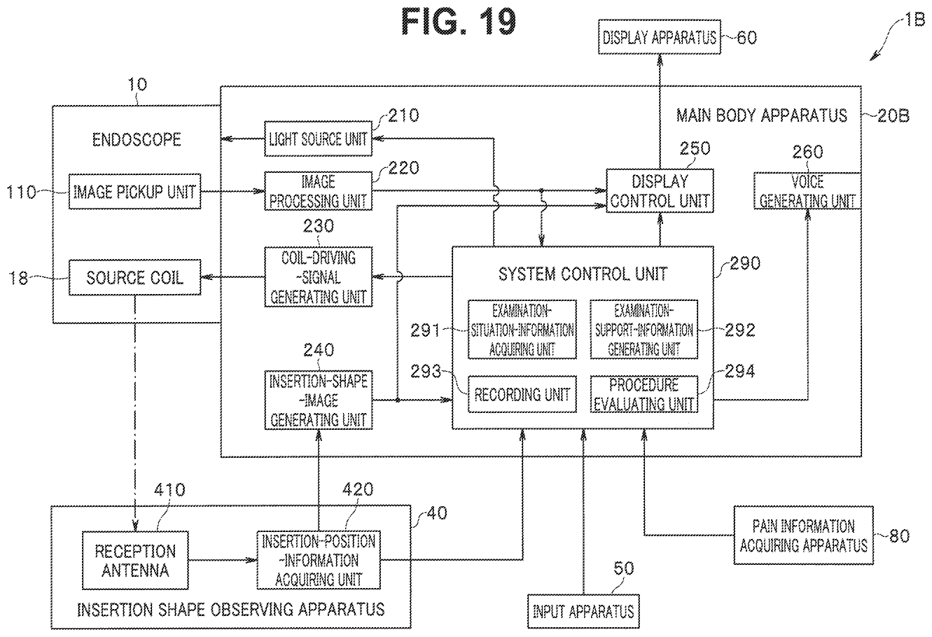

FIG. 19 to FIG. 22 relate to a third embodiment.

Note that, in the present embodiment, detailed explanation concerning portions having the same configurations and the like as the configurations and the like in at least one of the first embodiment or second embodiment is omitted as appropriate and portions having configurations and the like different from the configurations and the like in both of the first and second embodiments are mainly explained. In the present embodiment, an example is explained in which an endoscopic examination is performed on a patient equivalent to a subject.

An endoscope system 1B includes, for example, as shown in FIG. 19, the endoscope 10, a main body apparatus 20B, the insertion shape observing apparatus 40, the input apparatus 50, the display apparatus 60, and a pain information acquiring apparatus 80. FIG. 19 is a block diagram for explaining an example of a configuration of an endoscope system according to the third embodiment.

The main body apparatus 20B includes a system control unit 290 instead of the system control unit 270 in the main body apparatus 20. The main body apparatus 20B includes one or more processors 20P and the storage medium 20M.

The pain information acquiring apparatus 80 is configured to acquire pain information equivalent to information indicating an occurrence state of a pain of the patient during the endoscopic examination and output the acquired pain information to the main body apparatus 20B.

More specifically, the pain information acquiring apparatus 80 includes, as an apparatus for acquiring the pain information explained above, any one apparatus among, for example, a hand switch pressed by the patient in the endoscopic examination when the patient feels a pain, an electroencephalograph that measures a brain wave emitted from the patient, and a heart rate meter that measures a heart rate of the patient.

The system control unit 290 includes an examination-situation-information acquiring unit 291, an examination-support-information generating unit 292, a recording unit 293, and a procedure evaluating unit 294.

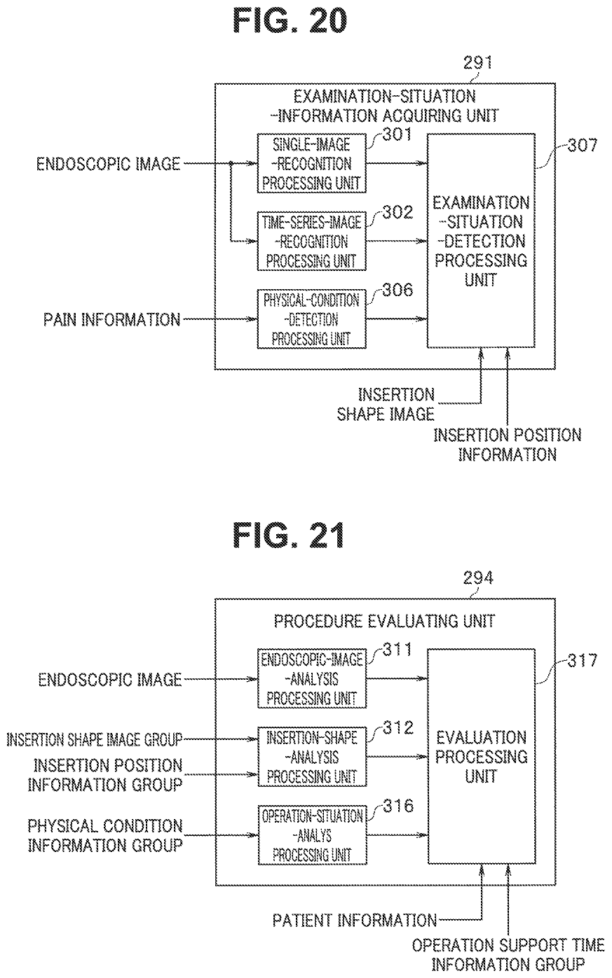

The examination-situation-information acquiring unit 291 is configured to perform, based on an endoscopic image outputted from the image processing unit 220, an insertion shape image outputted from the insertion-shape-image generating unit 240, insertion position information outputted from the insertion shape observing apparatus 40, and pain information outputted from the pain information acquiring apparatus 80, processing for acquiring examination situation information equivalent to information indicating a present examination situation in the endoscopic examination. More specifically, the examination-situation-information acquiring unit 291 includes, for example, as shown in FIG. 20, the single-image-recognition processing unit 301, the time-series-image-recognition processing unit 302, a physical-condition-detection processing unit 306, and an examination-situation-detection processing unit 307. FIG. 20 is a block diagram for explaining an example of a configuration of an examination-situation-information acquiring unit according to the third embodiment.

The physical-condition-detection processing unit 306 is configured to perform processing based on a detection result obtained by detecting the pain information outputted from the pain information acquiring apparatus 80 to thereby acquire physical condition information equivalent to information indicating a present physical condition of the patient during the endoscopic examination.

More specifically, the physical-condition-detection processing unit 306 is configured to be able to obtain, as information included in the physical condition information, at least one piece of information among, for example, presence or absence of occurrence of a pain of the patient or the number of times of occurrence of the pain of the patient during the endoscopic examination.

The examination-situation-detection processing unit 307 is configured to perform processing using at least one of the insertion position information outputted from the insertion shape observing apparatus 40, a recognition result obtained by the single-image-recognition processing unit 301, a recognition result obtained by the time-series-image-recognition processing unit 302, physical condition information obtained by the physical-condition-detection processing unit 306, or the insertion shape image outputted from the insertion-shape-image generating unit 240 to thereby acquire examination situation information indicating a present examination situation in the endoscopic examination.

The examination-support-information generating unit 292 is configured to perform, based on patient information inputted in the input apparatus 50, processing for determining whether insertion difficulty of the insertion section 11 in the patient subjected to the endoscopic examination is high.

Note that, in the present embodiment, as the patient information explained above, at least one piece of information among, for example, an age of the patent subjected to the endoscopic examination, sex of the patient, height of the patient, weight of the patient, a medical history of the patient, presence or absence of an experience of an endoscopic examination in the patient, a pain degree obtained at an endoscopic examination time in the past of the patient, or a model of an endoscope used at the endoscopic examination time in the past of the patient. In other words, in the present embodiment, only one or more pieces of information relating to insertion difficulty of the insertion section 11 in the patient subjected to the endoscopic examination have to be included in the patient information explained above. In the present embodiment, for example, the examination-support-information generating unit 292 may perform processing for acquiring the patient information explained above from an electronic clinical record system or the like outside the main body apparatus 20. According to the present embodiment, for example, when the patient subjected to the endoscopic examination underwent surgery of a digestive system in the past, a determination result that the insertion difficulty of the insertion section 11 in the patient is high only has to be obtained. According to the present embodiment, for example, when the patient subjected to the endoscopic examination did not undergo surgery of a digestive system in the past, a determination result that the insertion difficulty of the insertion section 11 in the patient is not high only has to be obtained.

The examination-support-information generating unit 292 is configured to, when obtaining the determination result that the insertion difficulty of the insertion section 11 in the patient subjected to the endoscopic examination is not high, based on examination situation information acquired by the examination-situation-information acquiring unit 291, perform processing for generating operation support information including guide information for normal difficulty and perform processing for outputting the generated operation support information to the display control unit 250 and the voice generating unit 260.

More specifically, the examination-support-information generating unit 292 is configured to, when obtaining the determination result that the insertion difficulty of the insertion section 11 in the patient subjected to the endoscopic examination is not high, read table data TDD for normal difficulty from the storage medium 20M, select, out of a plurality of pieces of guide information included in the read table data TDD, one guide information corresponding to a present examination situation specified based on the examination situation information acquired by the examination-situation-information acquiring unit 291, and generate operation support information including the selected one guide information and output the operation support information to the display control unit 250 and the voice generating unit 260.

The table data TDD for normal difficulty is created as data including the same configuration as the configuration of the table data TDA shown in FIG. 4.

The examination-support-information generating unit 292 is configured to, when obtaining the determination result that the insertion difficulty of the insertion section 11 in the patient subjected to the endoscopic examination is high, based on the examination situation information acquired by the examination-situation-information acquiring unit 291, perform processing for generating operation support information including guide information for high difficulty and perform processing for outputting the generated operation support information to the display control unit 250 and the voice generating unit 260.

More specifically, the examination-support-information generating unit 292 is configured to, when obtaining the determination result that the insertion difficulty of the insertion section 11 in the patient subjected to the endoscopic examination is high, read table data TDE for high difficulty from the storage medium 20M, select, out of a plurality of pieces of guide information included in the read table data TDE, one piece of guide information corresponding to a present examination situation specified based on the examination situation information acquired by the examination-situation-information acquiring unit 291, and generate operation support information including the selected one piece of guide information and output the operation support information to the display control unit 250 and the voice generating unit 260.

The table data TDE for high difficulty is created as data obtained by adding, as items respectively corresponding to the “examination situation” field, the “guide information” field, and the “parameter for operation check” field included in the table data TDA shown in FIG. 4, new items corresponding to factors (for example, adhesion of an intestinal tract and bending easiness of an insertion section) relating to an increase in insertion difficulty predicted from case data in the past.

In other words, the examination-support-information generating unit 292 is configured to generate, based on the examination situation information acquired by the examination-situation-information acquiring unit 291 and patient information including information relating to insertion difficulty of the insertion section 11 in the patient subjected to the endoscopic examination, operation support information for presenting different guide information according to whether the insertion difficulty of the insertion section 11 in the patient is high.

The recording unit 293 is configured to perform an operation for recording, as endoscopic examination information, an image, information, and the like obtained in the endoscopic examination.

More specifically, the recording unit 293 performs, during the endoscopic examination, an operation for respectively recording, for example, the endoscopic image outputted from the image processing unit 220, the insertion position information outputted from the insertion shape observing apparatus 40, the insertion shape image outputted from the insertion-shape-image generating unit 240, the physical condition information acquired by the physical-condition-detection processing unit 306, and the patient information inputted by operation of the input apparatus 50. The recording unit 293 performs, every time one operation support information is generated by the examination-support-information generating unit 292, operation for recording information indicating a time in which the one operation support information is continuously outputted to the display control unit 250.

In other words, the recording unit 293 records, as endoscopic examination information in one endoscopic examination, information correlating an endoscopic image group including endoscopic images for a plurality of frames sequentially outputted from the image processing unit 220 during the one endoscopic examination, an insertion position information group including a plurality of insertion position information sequentially outputted from the insertion shape observing apparatus 40 during the one endoscopic examination, an insertion shape image group including a plurality of insertion shape images sequentially outputted from the insertion-shape-image generating unit 240 during the one endoscopic examination, a physical condition information group including a plurality of pieces of physical condition information acquired according to an occurrence state of a pain of the patient during the one endoscopic examination, an operation support time information group indicating output times and output contents of a respective plurality of pieces of operation support information outputted from the examination-support-information generating unit 292 to the display control unit 250 during the one endoscopic examination, and patient information, which is information used for determination of insertion difficulty of the insertion section 11 in the patient subjected to the one endoscopic examination.

The procedure evaluating unit 294 is configured to, based on the endoscopic examination information recorded by the recording unit 293, generate procedure evaluation information including information obtained by evaluating a procedure including inserting operation for the insertion section 11 performed by the user during the endoscopic examination and perform processing for outputting the generated procedure evaluation information to the display control unit 250. More specifically, the procedure evaluating unit 294 includes, for example, as shown in FIG. 21, the endoscopic-image-analysis processing unit 311, the insertion-shape-analysis processing unit 312, a physical-condition-analysis processing unit 316, and an evaluation processing unit 317. FIG. 21 is a block diagram for explaining an example of a configuration of a procedure evaluating unit according to the third embodiment.

The physical-condition-analysis processing unit 316 is configured to perform analysis processing based on the physical condition information group included in the endoscopic examination information recorded by the recording unit 293 to thereby obtain an analysis result including analysis values of one or more evaluation indicators corresponding to a physical condition of the patient during the endoscopic examination.

More specifically, by performing the analysis processing based on the operation situation information group included in the endoscopic examination information recorded by the recording unit 293, the physical-condition-analysis processing unit 316 can obtain an analysis result including an analysis value of at least one evaluation indicator among, for example, an occurrence frequency of a pain of the patient, strength of the pain of the patient, or an occurrence time of the pain of the patient during the endoscopic examination.

The evaluation processing unit 317 is configured to perform processing using an analysis result obtained by the endoscopic-image-analysis processing unit 311, an analysis result obtained by the insertion-shape-analysis processing unit 312, an analysis result obtained by the operation-situation-analysis processing unit 314, and the operation support time information group and the patient information included in the endoscopic examination information recorded by the recording unit 293 to thereby generate procedure evaluation information and perform processing for outputting the generated procedure evaluation information to the display control unit 250.

In the present embodiment, at least a part of functions of the main body apparatus 20B only has to be realized by the processor 20P. In the present embodiment, at least a part of the main body apparatus 20B may be configured as individual electronic circuits or may be configured as a circuit block in an integrated circuit such as an FPGA (field programmable gate array). By modifying the configuration according to the present embodiment as appropriate, for example, a computer may read, from the storage medium 20M such as a memory, a program for executing at least a part of the functions of the main body apparatus 20B and perform an operation corresponding to the read program.

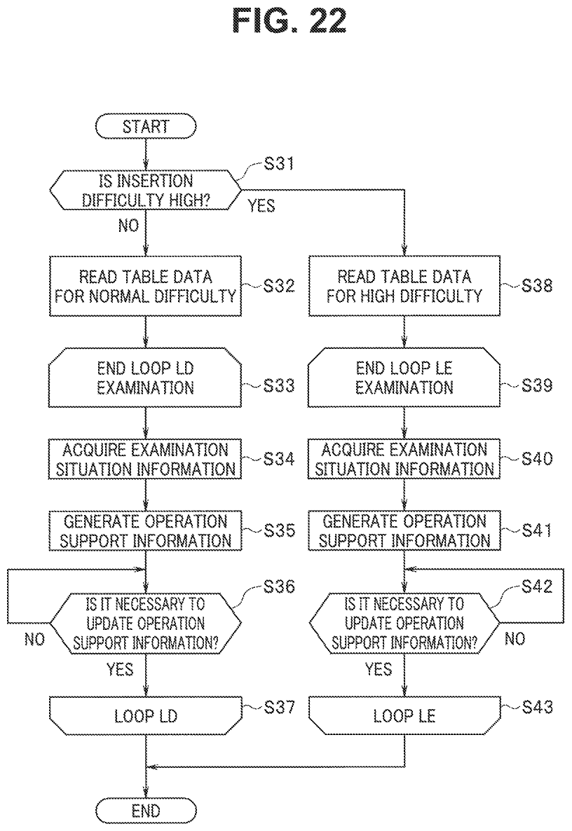

Subsequently, action of the present embodiment is explained with reference to FIG. 22. FIG. 22 is a flowchart for explaining an example of processing performed in the endoscope system according to the third embodiment.

After connecting the respective sections of the endoscope system 1B and turning on the endoscope system 1B, the user operates the input apparatus 50 to thereby input patient information used for determination of insertion difficulty of the insertion section 11 in the patient subjected to the endoscopic examination.

With the operation of the user explained above, an object is irradiated with illumination light supplied from the light source unit 210, an image of the object irradiated with the illumination light is picked up by the image pickup unit 110, and an endoscopic image obtained by picking up an image of the object is outputted from the image processing unit 220 to the display control unit 250 and the system control unit 290. With the operation of the user explained above, a coil driving signal is supplied from the coil-driving-signal generating unit 230, magnetic fields are emitted from the respective plurality of source coils 18 according to the coil driving signal, insertion position information obtained by detecting the magnetic fields is outputted from the insertion-position-information acquiring unit 420 to the insertion-shape-image generating unit 240 and the system control unit 290, and an insertion shape image generated according to the insertion position information is outputted from the insertion-shape-image generating unit 240 to the system control unit 290.

The examination-support-information generating unit 292 performs, based on the patient information inputted in the input apparatus 50, processing for determining whether insertion difficulty of the insertion section 11 in the patient subjected to the endoscopic examination is high (step S31 in FIG. 22).

When obtaining a determination result that the insertion difficulty of the insertion section 11 in the patient subjected to the endoscope examination is not high (S31: NO), after reading the table data TDD for normal difficulty from the storage medium 20M (step S32 in FIG. 22) and performing processing for causing the recording unit 293 to record information indicating the determination result together with physical condition information, the examination-support-information generating unit 292 shifts to processing in step S33 in FIG. 22 explained below. When obtaining a determination result that the insertion difficulty of the insertion section 11 in the patient subjected to the endoscopic examination is high (S31: YES), after reading the table data TDE for high difficulty from the storage medium 20M (step S38 in FIG. 22) and performing processing for causing the recording unit 293 to record information indicating the determination result together with the physical condition information, the examination-support-information generating unit 292 shifts to processing in step S39 in FIG. 22 explained below.

After inputting patient information, the user starts operation for inserting the insertion section 11 from an anus into an inside of a large intestine of a subject. For example, immediately after starting the insertion of the insertion section 11 into the large intestine of the patient, the user operates the examination start switch of the input apparatus 50 to thereby perform an instruction for starting an operation relating to support of an endoscopic examination for the subject.

With the operation of the user explained above, pain information indicating an occurrence state of a pain of the patient during the endoscopic examination is outputted from the pain information acquiring apparatus 80 to the main body apparatus 20B.

When detecting an instruction from the examination start switch of the input apparatus 50 after reading the table data TDD for normal difficulty, the examination-situation-information acquiring unit 291 and the examination-support-information generating unit 292 start processing of a loop LD, which is processing for performing operation support corresponding to a present examination situation in the endoscopic examination (step S33 in FIG. 22).

The examination-situation-information acquiring unit 291 performs, based on the endoscopic image outputted from the image processing unit 220, the insertion shape image outputted from the insertion-shape-image generating unit 240, the insertion position information outputted from the insertion shape observing apparatus 40, and the pain information outputted from the pain information acquiring apparatus 80, processing for acquiring examination situation information equivalent to information indicating a present examination situation in the endoscopic examination (step S34 in FIG. 22).

The examination-support-information generating unit 292, based on the examination situation information acquired by the examination-situation-information acquiring unit 291 and the table data TDD read in step S32 in FIG. 22, performs processing for generating operation support information and performs processing for outputting the generated operation support information to the display control unit 250 and the voice generating unit 260 (step S35 in FIG. 22).

For example, the display control unit 250, based on the endoscopic image outputted from the image processing unit 220 and the operation support information outputted from the system control unit 290, performs processing for generating a display image including the same information as the information included in the display image DGA shown in FIG. 7 and performs processing for causing the display apparatus 60 to display the generated display image.

The voice generating unit 260 generates voice corresponding to guide information included in the operation support information outputted from the system control unit 290 and performs an operation for outputting the generated voice to the outside of the main body apparatus 20B at every predetermined time.

The examination-support-information generating unit 292 performs, based on the examination situation information acquired by the examination-situation-information acquiring unit 291, for example, every time an endoscopic image for one frame is outputted from the image processing unit 220, processing for determining whether it is necessary to update the operation support information generated in step S35 in FIG. 22 (currently being outputted) (step S36 in FIG. 22).

Note that, in the present embodiment, the same processing as the processing in step S4 in FIG. 6 only has to be performed as the processing in step S36 in FIG. 22.

When obtaining a determination result that it is necessary to update the operation support information generated in step S35 in FIG. 22 (S36: YES), after performing processing for causing the recording unit 293 to record, together with operation support time information, information indicating an estimation result obtained by estimating whether operation corresponding to the guide information included in the operation support information is completed, the examination-support-information generating unit 292 performs processing in step S37 in FIG. 22 explained below. When obtaining a determination result that it is unnecessary to update the operation support information generated in step S35 in FIG. 22 (S36: NO), after outputting the operation support information to the display control unit 250 and the voice generating unit 260 again, the examination-support-information generating unit 292 performs the processing in step S36 in FIG. 22 again.

Note that it is assumed that the examination-support-information generating unit 292 shifts to the processing in step S37 in FIG. 22 explained below not only when obtaining the determination result that it is necessary to update the operation support information generated in step S35 in FIG. 22 but also, for example, when detecting that a predetermined time has elapsed after acquiring the determination result that it is unnecessary to update the operation support information or detecting that the processing in step S36 in FIG. 22 is performed a predetermined number of times.

For example, immediately after completing removal of the insertion section 11 inserted into the large intestine of the subject, by operating the examination end switch of the input apparatus 50, the user performs an instruction for stopping the operation relating to the support of the endoscopic examination for the subject.

The examination-situation-information acquiring unit 291 and the examination-support-information generating unit 292 perform termination processing for the loop LD (step S37 in FIG. 22). More specifically, for example, when failing in detecting the instruction from the examination end switch of the input apparatus 50, the examination-situation-information acquiring unit 291 and the examination-support-information generating unit 292 return to step S33 in FIG. 22 and perform the processing of the loop LD again. For example, when detecting the instruction from the examination end switch of the input apparatus 50, the examination-situation-information acquiring unit 291 and the examination-support-information generating unit 292 end the processing of the loop LD and end a series of processing shown in FIG. 22.

When detecting the instruction from the examination start switch of the input apparatus 50 after reading the table data TDE for high difficulty, the examination-situation-information acquiring unit 291 and the examination-support-information generating unit 292 start processing of a loop LE, which is processing for performing operation support corresponding to a present examination situation in the endoscopic examination (step S39 in FIG. 22).

The examination-situation-information acquiring unit 291 performs, based on the endoscopic image outputted from the image processing unit 220, the insertion shape image outputted from the insertion-shape-image generating unit 240, the insertion position information outputted from the insertion shape observing apparatus 40, and the pain information outputted from the pain information acquiring apparatus 80, processing for acquiring examination situation information equivalent to information indicating a present examination situation in the endoscopic examination (step S40 in FIG. 22).

The examination-support-information generating unit 292, based on the examination situation information acquired by the examination-situation-information acquiring unit 291 and the table data TDE read in step S38 in FIG. 22, performs processing for generating operation support information and performs processing for outputting the generated operation support information to the display control unit 250 and the voice generating unit 260 (step S41 in FIG. 22).

The display control unit 250, based on the endoscopic image outputted from the image processing unit 220 and the operation support information outputted from the system control unit 290, performs processing for generating a display image DGE that can include information different from the information included in the display image DGA shown in FIG. 7 and performs processing for causing the display apparatus 60 to display the generated display image DGE.

The display image DGE is generated as, for example, an image in which, in respective regions of the general situation display region GDB, the guide type display region GDC, and the guide content display region GDD, one of the same information as the information displayed in the display image DGA shown in FIG. 7 and information corresponding to a phenomenon (for example, adhesion of an intestinal tract) that can occur when the insertion difficulty of the insertion section 11 is high is selectively displayed.

The examination-support-information generating unit 292 performs, based on the examination situation information acquired by the examination-situation-information acquiring unit 291, for example, every time an endoscopic image for one frame is outputted from the image processing unit 220, processing for determining whether it is necessary to update the operation support information generated in step S41 in FIG. 22 (currently being outputted) (step S42 in FIG. 22).

Note that, in the present embodiment, the same processing as the processing in step S4 in FIG. 6 only has to be performed as the processing in step S42 in FIG. 22.

When obtaining the determination result that it is necessary to update the operation support information generated in step S41 in FIG. 22 (S42: YES), the examination-support-information generating unit 292 performs processing in step S43 in FIG. 22 explained below. When obtaining the determination result that it is unnecessary to update the operation support information generated in step S41 in FIG. 22 (S42: NO), after outputting the operation support information to the display control unit 250 and the voice generating unit 260 again, the examination-support-information generating unit 292 performs the processing in step S42 in FIG. 22 again.

Note that it is assumed that the examination-support-information generating unit 292 shifts to the processing in step S43 in FIG. 22 explained below not only when obtaining the determination result that it is necessary to update the operation support information generated in step S41 in FIG. 22 but also, for example, when detecting that a predetermined time has elapsed after acquiring the determination result that it is unnecessary to update the operation support information or detecting that the processing in step S42 in FIG. 22 is performed a predetermined number of times.

For example, immediately after completing removal of the insertion section 11 inserted into the large intestine of the subject, by operating the examination end switch of the input apparatus 50, the user performs an instruction for stopping the operation relating to the support of the endoscopic examination for the subject.

The examination-situation-information acquiring unit 291 and the examination-support-information generating unit 292 perform termination processing for the loop LE (step S43 in FIG. 22). More specifically, for example, when failing in detecting the instruction from the examination end switch of the input apparatus 50, the examination-situation-information acquiring unit 291 and the examination-support-information generating unit 292 return to step S39 in FIG. 22 and perform the processing of the loop LE again. For example, when detecting the instruction from the examination end switch of the input apparatus 50, the examination-situation-information acquiring unit 291 and the examination-support-information generating unit 292 end the processing of the loop LE and end the series of processing shown in FIG. 22.

For example, at any timing after operating the examination end switch of the input apparatus 50, the user operates the procedure evaluation display switch of the input apparatus 50 to thereby perform an instruction for displaying an evaluation result of a procedure performed during the endoscopic examination.

When detecting the instruction from the procedure evaluation display switch of the input apparatus 50, the procedure evaluating unit 294 performs processing for determining in which of a normal difficulty time and a high difficulty time the endoscopic examination information recorded by the recording unit 293 is obtained.

When obtaining a determination result that the endoscopic examination information recorded by the recording unit 293 is obtained at the normal difficulty time, the procedure evaluating unit 294 generates procedure evaluation information for normal difficulty and outputs the procedure evaluation information for normal difficulty to the display control unit 250.

More specifically, when obtaining the determination result that the endoscopic examination information recorded by the recording unit 293 is obtained at the normal difficulty time, for example, the evaluation processing unit 317 of the procedure evaluating unit 294 performs an arithmetic operation using table data TDQ indicating a correspondence relation between a deviation value and a score at the normal difficulty time to thereby calculate a procedure score and generates procedure evaluation information including the calculated procedure score and outputs the procedure evaluation information to the display control unit 250. Note that the table data TDQ explained above only has to be created as, for example, the same data as the table data TDP shown in FIG. 13.

The display control unit 250, based on the procedure evaluation information for normal difficulty outputted from the procedure evaluating unit 294, for example performs processing for generating a display image including the same information as the information included in the display image DGB shown in FIG. 12 and performs processing for causing the display apparatus 60 to display the generated display image.

When obtaining a determination result that the endoscopic examination information recorded by the recording unit 293 is obtained at the high difficulty time, the procedure evaluating unit 294 generates the procedure evaluation information for high difficulty and outputs the procedure evaluation information for high difficulty to the display control unit 250.

More specifically, when obtaining the determination result that the endoscopic examination information recorded by the recording unit 293 is obtained at the high difficulty time, for example, the evaluation processing unit 317 of the procedure evaluating unit 294 reads, from the storage medium 20M, table data TDR indicating a correspondence relation between a deviation value and a score at the high difficulty time, performs an arithmetic operation using the read table data TDR to thereby calculate a procedure score, and generates procedure evaluation information including the calculated procedure score and outputs the procedure evaluation information to the display control unit 250. Note that the table data TDR explained above only has to be created as, for example, data from which a score equal to or higher than one score calculated using a calculation method for the table data TDQ is obtained in a range of the same deviation value as a range of one deviation value included in the table data TDQ.

Note that, according to the present embodiment, for example, when detecting, based on at least one of an analysis result obtained by the endoscopic-image-analysis processing unit 311 or an analysis result obtained by the insertion-shape-analysis processing unit 312, a predetermined insertion difficult situation such as a situation in which an amount of a residue in an intestinal tract for which the endoscopic examination is performed is large, the evaluation processing unit 317 may invalidate the determination result relating to the insertion difficulty of the insertion section 11 recorded by the recording unit 293 according to the processing in step S31 in FIG. 22 and acquire, anew, a determination result that the endoscopic examination information recorded by the recording unit 293 is obtained at the high difficulty time.

The display control unit 250, based on the procedure evaluation information for high difficulty outputted from the procedure evaluating unit 294, performs processing for generating a display image DGF that can include information different from the information included in the display image DGB shown in FIG. 12 and performs processing for causing the display apparatus 60 to display the generated display image DGF.

The display image DGF is generated as, for example, an image in which, in the comment display region DDA, at least one of the same comment as the comment displayed in the display image DGB shown in FIG. 12 or a comment corresponding to a phenomenon (for example, adhesion of an intestinal tract) that can occur when the insertion difficulty of the insertion section 11 is high.

As explained above, according to the present embodiment, it is possible to present an operation method for the insertion section 11 corresponding to insertion difficulty of the insertion section 11 in a patient subjected to an endoscopic examination and a present examination situation in the endoscopic examination. According to the present embodiment, it is possible to present an evaluation for a procedure performed during the endoscopic examination to a user after an end of the endoscopic examination. Accordingly, according to the present embodiment, it is possible to further improve examination quality in the endoscopic examination than in the past.

Note that, according to the present embodiment, for example, the examination-support-information generating unit 292 may be configured to, when detecting that information capable of specifying a respective plurality of patients is included in the patient information inputted in the input apparatus 50, perform processing for causing the recording unit 293 to record endoscopic examination information obtained during the endoscopic examination while distinguishing the endoscopic examination information for each of the patients. Further, according to the present embodiment, for example, the system control unit 290 may be configured to perform an operation for outputting the endoscopic examination information, which is recorded by the recording unit 293 in a state in which the endoscopic examination information is distinguished for each of the patients, to a database or a Cloud system outside the main body apparatus 20B.

By combining the configuration of the second embodiment and the configuration of the present embodiment as appropriate, for example, processing for generating operation support information corresponding to examination situation information indicating a present examination situation in an endoscopic examination, the user information explained in the second embodiment, and the patient information explained in the present embodiment may be performed.

By combining the configuration of the second embodiment and the configuration of the present embodiment as appropriate, for example, processing for generating procedure evaluation information corresponding to an analysis result obtained by analysis processing based on an endoscopic image group recorded during one endoscopic examination, an analysis result obtained by analysis processing based on an insertion shape image group and an insertion position information group recorded during the one endoscopic examination, an analysis result obtained by analysis processing based on an operation situation information group recorded during the one endoscopic examination, an analysis result obtained by analysis processing based on a physical condition information group recorded during the one endoscopic examination, and an operation support time information group and subject information recorded during the one endoscopic examination may be performed.

Note that the present invention is not limited to the respective embodiments explained above. It goes without saying that various changes and applications are possible within a range not departing from the gist of the invention.

What is claimed is:

1. An endoscopic examination supporting apparatus comprising at least one processor including hardware, wherein
the processor acquires insertion shape information indicating an insertion shape of an insertion section of an endoscope inserted into a subject, evaluates, based on the insertion shape information, a procedure including inserting operation for the insertion section performed by a user who operates the endoscope during an endoscopic examination, and generates procedure evaluation information.

2. The endoscopic examination supporting apparatus according to claim 1, wherein
the processor
acquires, based on at least one endoscopic image obtained by picking up an image of an inside of the subject with the endoscope and the insertion shape information, examination situation information indicating an examination situation in the endoscopic examination performed on the subject using the endoscope,
generates, based on the examination situation information, operation support information for supporting the inserting operation for the insertion section performed by the user who operates the endoscope in the endoscopic examination, and
generates the procedure evaluation information based on the examination situation information.

3. The endoscopic examination supporting apparatus according to claim 2, further comprising a recording unit configured to record, as endoscopic examination information, an endoscopic image group including the endoscopic image in plurality obtained during the endoscopic examination, an insertion shape information group including the insertion shape information in plurality, and an operation support time information group including the operation support information in plurality in correlation with one another, wherein
the processor generates the procedure evaluation information based on the endoscopic examination information recorded by the recording unit.

4. The endoscopic examination supporting apparatus according to claim 2, wherein
the processor
performs processing for recognizing an examination situation corresponding to one of the endoscopic images and obtaining a first recognition result,
performs processing for recognizing an examination situation corresponding to the plurality of endoscopic images that are temporally consecutive and obtaining a second recognition result, and
acquires the examination situation information by performing processing using at least one of the first recognition result, the second recognition result, insertion position information, which is information included in the insertion shape information and is information indicating an insertion position of the insertion section inserted into the subject, or an insertion shape image, which is information included in the insertion shape information and is information obtained by visualizing an insertion shape of the insertion section inserted into the subject.

5. The endoscopic examination supporting apparatus according to claim 2, further comprising a recording unit configured to record, as endoscopic examination information, an endoscopic image group including the endoscopic image in plurality obtained during the endoscopic examination, an insertion shape information group including the insertion shape information in plurality, and an operation support time information group including the operation support information in plurality in correlation with one another, wherein
the operation support time information group includes information capable of specifying, for each piece of guide information, a presentation time when a plurality of pieces of guide information included in a respective plurality of pieces of operation support information generated during the endoscopic examination are presented to the user, and
the processor
performs analysis processing based on the endoscopic image group included in the endoscopic examination information to thereby obtain a first analysis result including analysis values of one or more evaluation indicators corresponding to operation of the endoscope estimated to be performed by the user during the endoscopic examination,
performs the analysis processing based on the insertion shape information group including the insertion shape information in plurality included in the endoscopic examination information to thereby obtain a second analysis result including analysis values of one or more evaluation indicators corresponding to the operation of the endoscope estimated to be performed by the user during the endoscopic examination, and
generates the procedure evaluation information by performing processing using the first analysis result, the second analysis result, and the operation support time information group included in the endoscopic examination information.

6. The endoscopic examination supporting apparatus according to claim 2, further comprising a recording unit configured to record, as endoscopic examination information, an endoscopic image group including the endoscopic image in plurality obtained during the endoscopic examination, an insertion shape information group including the insertion shape information in plurality, and an operation support time information group including the operation support information in plurality in correlation with one another, wherein
the processor performs processing for causing the recording unit to record information indicating an estimation result obtained by estimating whether operation corresponding to guide information included in the operation support information is completed.

7. The endoscopic examination supporting apparatus according to claim 6, wherein the processor calculates, based on information indicating the estimation result recorded by the recording unit, an operation completion ratio equivalent to a rate of operation corresponding to each piece of guide information presented during the endoscopic examination being completed and generates the procedure evaluation information including information indicating the calculated operation completion ratio.

8. The endoscopic examination supporting apparatus according to claim 1, wherein the processor calculates a procedure score indicating a comprehensive evaluation of a procedure performed during the endoscopic examination and generates the procedure evaluation information including the calculated procedure score.

9. The endoscopic examination supporting apparatus according to claim 1, further comprising a recording unit configured to record endoscopic examination information obtained during the endoscopic examination, wherein
the processor calculates, based on the endoscopic examination information recorded by the recording unit, an examination evaluation value used to evaluate quality of the endoscopic examination.

10. The endoscopic examination supporting apparatus according to claim 9, wherein the processor calculates, as the examination evaluation value, at least any one of an ileocecum reaching ratio, an intestinal tract cleaning degree, or an adenoma detection rate.

11. The endoscopic examination supporting apparatus according to claim 2, wherein the processor performs processing for generating visual information corresponding to the operation support information and causing a display apparatus to display the visual information and performs processing for generating visual information corresponding to the procedure evaluation information and causing the display apparatus to display the visual information.

12. The endoscopic examination supporting apparatus according to claim 2, wherein the processor generates and outputs voice corresponding to the operation support information.

13. The endoscopic examination supporting apparatus according to claim 2, wherein the processor presents the operation support information in a different pattern for each operation difficulty relating to the inserting operation for the insertion section.

14. The endoscopic examination supporting apparatus according to claim 4, wherein
the processor acquires operation situation information equivalent to information indicating a present operation situation of the user who is operating the endoscope, and
acquires the examination situation information by performing processing using at least one of the first recognition result, the second recognition result, the insertion position information, the insertion shape image, or the operation situation information.

15. The endoscopic examination supporting apparatus according to claim 14, wherein the processor acquires the operation situation information by performing processing based on a detection result obtained by detecting at least one of a video obtained by photographing the user, motion information capable of specifying a motion of the user, visual line information capable of specifying a visual line of the user, or voice acquired from the user.

16. The endoscopic examination supporting apparatus according to claim 14, wherein the processor generates, based on the examination situation information and user information including information relating to a skill level of the user who operates the endoscope, the operation support information for presenting different guide information according to whether the skill level of the user is equivalent to an expert skill level.

17. The endoscopic examination supporting apparatus according to claim 16, wherein the user information including the information relating to the skill level of the user who operates the endoscope includes at least one piece of information among a number of years of experience of the endoscopic examination in the user, a number of experienced cases in the user, a diagnosis and treatment department of the user, presence or absence of a certified physician qualification in the user, or presence or absence of a specialist qualification in the user.

18. The endoscopic examination supporting apparatus according to claim 14, wherein, when obtaining, based on the user information including information relating to a skill level of the user who operates the endoscope, a determination result that a skill level of the user is equivalent to an expert skill level, the processor performs processing for generating the operation support information including guide information for presenting work content currently performed by the user.

19. The endoscopic examination supporting apparatus according to claim 14, further comprising a recording unit configured to record endoscopic examination information obtained during the endoscopic examination, wherein
the recording unit records, as the endoscope examination information, an endoscopic image group including the endoscopic image in plurality obtained during the endoscopic examination, an insertion shape information group including the insertion shape information in plurality, an operation support time information group including the operation support information in plurality, and an operation situation information group including a plurality of pieces of operation situation information acquired according to an action of the user in the endoscopic examination in correlation with one another.

20. The endoscopic examination supporting apparatus according to claim 19, wherein
the processor
performs analysis processing based on the endoscopic image group included in the endoscopic examination information to thereby obtain a first analysis result including analysis values of one or more evaluation indicators corresponding to operation of the endoscope estimated to be performed by the user during the endoscopic examination,
performs the analysis processing based on the insertion shape information group including the insertion shape information in plurality included in the endoscopic examination information to thereby obtain a second analysis result including analysis values of one or more evaluation indicators corresponding to the operation of the endoscope estimated to be performed by the user during the endoscopic examination,
performs analysis processing based on the operation situation information group included in the endoscopic examination information to thereby obtain a third analysis result including analysis values of one or more evaluation indexes corresponding to an action of the user during the endoscopic examination, and
acquires the procedure evaluation information by performing processing using the first analysis result, the second analysis result, the third analysis result, and the operation support time information group.

21. The endoscopic examination supporting apparatus according to claim 4, wherein
the processor acquires physical condition information equivalent to information indicating a present physical condition of the subject during the endoscopic examination, and
acquires the examination situation information by performing processing using at least one of the first analysis result, the second analysis result, the insertion position information, the insertion shape image, or the physical condition information.

22. The endoscopic examination supporting apparatus according to claim 21, wherein processor acquires the physical condition information by performing processing based on a detection result obtained by detecting pain information equivalent to information indicating an occurrence state of a pain of the subject during the endoscopic examination.

23. The endoscopic examination supporting apparatus according to claim 22, wherein the pain information is information acquired by at least any one of a hand switch pressed by the subject when feeling a pain during the endoscopic examination, an electroencephalograph that measures a brain wave emitted from the subject, or a heart rate meter that measures a heart rate of the subject.

24. The endoscopic examination supporting apparatus according to claim 21, wherein the processor presents, based on the examination situation information and subject information including information relating to insertion difficulty of the insertion section in the subject, different guide information according to whether the insertion difficulty of the insertion section in the subject is high.

25. The endoscopic examination supporting apparatus according to claim 24, wherein the subject information includes at least one piece of information among an age of the subject, sex of the subject, height of the subject, weight of the subject, a medical history of the subject, presence or absence of an experience of the endoscopic examination in the subject, a pain degree obtained at an endoscopic examination time in past of the subject, or a model of an endoscope used at the endoscopic examination time in the past of the subject.

26. The endoscopic examination supporting apparatus according to claim 24, further comprising a recording unit configured to record endoscopic examination information obtained during the endoscopic examination, wherein
the recording unit is configured to record, as the endoscopic examination information, an endoscopic image group including the endoscopic image in plurality obtained during the endoscopic examination, an insertion shape information group including the insertion shape information in plurality, an operation support time information group including the operation support information in plurality, the subject information, and a physical condition information group including a plurality of pieces of physical condition information acquired according to an occurrence state of a pain of the subject during the endoscopic examination in correlation with one another.

27. The endoscopic examination supporting apparatus according to claim 26, wherein the processor
performs analysis processing based on the endoscopic image group included in the endoscopic examination information to thereby obtain a first analysis result including analysis values of one or more evaluation indicators corresponding to operation of the endoscope estimated to be performed by the user during the endoscopic examination,
performs the analysis processing based on the insertion shape information group including the insertion shape information in plurality included in the endoscopic examination information to thereby obtain a second analysis result including analysis values of one or more evaluation indicators corresponding to the operation of the endoscope estimated to be performed by the user during the endoscopic examination,
performs analysis processing based on the physical condition information group included in the endoscopic examination information to thereby obtain a third analysis result including analysis values of one or more evaluation indicators corresponding to a physical condition of a patient during the endoscopic examination, and
acquires the procedure evaluation information by performing processing using the first analysis result, the second analysis result, the third analysis result, the operation support time information group, and the subject information.

28. An endoscopic examination supporting method comprising:
acquiring insertion shape information indicating an insertion shape of an insertion section of an endoscope inserted into a subject; and
evaluating, based on the insertion shape information, a procedure including inserting operation for the insertion section performed by a user who operates the endoscope during an endoscopic examination and generating procedure evaluation information.

29. A non-transitory recording medium recording a program for causing a computer to execute processing for:
acquiring insertion shape information indicating an insertion shape of an insertion section of an endoscope inserted into a subject; and
evaluating, based on the insertion shape information, a procedure including inserting operation for the insertion section performed by a user who operates the endoscope during an endoscopic examination and generating procedure evaluation information.
